(19) United States
(12) Patent Application Publication
(10) Pub. No.: US 2026/0283577 A1
(43) Pub. Date: Sep. 24, 2026

(54) ENAMEL WIDTH AND DENTIN SHAPE FROM NEAR INFRARED SCANS

(71) Applicant: ALIGN TECHNOLOGY, INC., San Jose, CA (US)

(72) Inventors: Maayan MOSHE, Ramat Hasharon (IL); Shai AYAL, Tel Aviv (IL); Doron MALKA, Tel Aviv (IL); Ran KATZ, Hod Hasharon (IL)

(21) Appl. No.: 19/574,093

(22) Filed: Mar. 20, 2026

Related U.S. Application Data
(60) Provisional application No. 63/775,727, filed on Mar. 21, 2025.

(51) Int. Cl. A61B 6/51; A61C 9/00; G06T 7/00; G06T 7/13

(52) U.S. Cl. CPC A61B 6/512; A61C 9/0053; G06T 7/0012; G06T 7/13; G06T 2207/10008; G06T 2207/10048; G06T 2207/20081; G06T 2207/30036

(57) ABSTRACT

Systems, apparatuses, and methods disclosed herein are directed to systems, methods, and apparatuses for enabling the determination of enamel width and dentin shape and location from near infrared and 3D surface scan data for use in treating a patient’s dentition.

### RELATED APPLICATIONS

This application claims the benefit under 35 U.S.C. §119(e) of U.S. Provisional Patent Application No. 63/775,727, filed Mar. 21, 2025, and titled “Enamel Width and Dentin Shape from Near Infrared Scans,” which is incorporated herein, in its entirety, by this reference.

### BACKGROUND

Determining the location and geometry of dentin and enamel is useful for effective dental treatment planning because it directly influences restorative and preventive strategies. The enamel serves as the outermost protective layer of a tooth, while the dentin, located beneath it, provides structural support for the tooth and houses the pulp. Accurate knowledge of these anatomical features helps dentists assess the extent of decay, plan conservative cavity preparations, and optimize restorative materials. For example, in restorative procedures like fillings or crowns, knowing the enamel thickness ensures minimal removal of healthy tissue, preserving tooth integrity.

Despite its importance, current methods for detecting dentin location, geometry, and enamel width have notable shortcomings. Traditional diagnostic tools like radiographs provide only two-dimensional images, limiting their accuracy in assessing enamel thickness and dentin topography. Cone-beam computed tomography offers improved visualization but is not always accessible due to its high cost and radiation exposure concerns. Optical coherence tomography and other advanced imaging techniques can enhance precision but are still not widely implemented in routine clinical practice due to equipment costs and technical limitations. Additionally, indirect methods, such as probing or transillumination, rely on practitioner expertise and may lead to inconsistent results.

What is desired are systems, apparatuses, and methods for more effectively enabling dentin and enamel detection. Embodiments of the disclosure address this and other objectives both individually and collectively.

### SUMMARY

In some embodiments, the systems, apparatuses, and methods disclosed and/or described herein are directed to systems, methods, and apparatuses for detecting dentin and enamel.

In some embodiments, a method for use in dental treatment planning my include receiving or collecting a plurality of two-dimensional (2D) near infrared intraoral scanner images of a patient’s tooth and three-dimensional (3D) surface data of the patient’s tooth, receiving a parameterized dentin template, identifying, for each image of the plurality of 2D intraoral scanner images, a border between a dentin and an enamel in the images using a detection algorithm that uses the plurality of 2D near infrared images, and projecting the border between the dentin and the enamel identified in each image onto a three-dimensional (3D) model of the patient’s tooth. The 3D model of the patient’s tooth may be generated from 3D surface data of the patient’s tooth. The method may also include iteratively projecting the dentin template on the 3D model for each of the 2D images until the projected template aligns with the border between the dentin and the enamel identified in each image, generating a 3D model of the dentin based on the projection, and outputting the 3D model of the dentin.

In some embodiments, a method for use in dental treatment planning may include receiving or collecting a plurality of two-dimensional (2D) near infrared intraoral scanner images of a patient’s tooth and three-dimensional (3D) surface data of the patient’s tooth, identifying, for each image of the plurality of 2D intraoral scanner images, a border between a dentin and an enamel in the images using a detection algorithm that uses the plurality of 2D near infrared images, and projecting the border between the dentin and the enamel identified in each image onto a three-dimensional (3D) model of the patient’s tooth. The 3D model of the patient’s tooth may be generated from 3D surface data of the patient’s tooth. The method may also include correcting the location of the border between the enamel and the dentin by tracing rays between a modeled camera and the 3D model of the tooth, generating a 3D model of the dentin based on the projection, and outputting the 3D model of the dentin.

In some embodiments, a method for use in dental treatment planning may include receiving or collecting a plurality of two-dimensional (2D) near infrared intraoral scanner images of a patient’s tooth and three-dimensional (3D) surface data of the patient’s tooth, identifying, for each image of the plurality of 2D intraoral scanner images, a border between a dentin and an enamel in the images using a detection algorithm that uses the plurality of 2D near infrared images, and projecting the border between the dentin and the enamel identified in each image onto a three-dimensional (3D) model of the patient’s tooth. The 3D model of the patient’s tooth may be generated from 3D surface data of the patient’s tooth. The method may also include detecting an outer edge of the tooth relative to the border between a dentin and an enamel based on a location of the outer edge of the tooth of the 3D model in the projection, determining an enamel width for the patient’s tooth based on the projected border, and outputting an indicator of enamel width. Other objects and advantages of the systems, apparatuses, and methods disclosed will be apparent to one of ordinary skill in the art upon review of the detailed description and the included figures. Throughout the drawings, identical reference characters and descriptions indicate similar, but not necessarily identical, elements. While the embodiments disclosed or described herein are susceptible to various modifications and alternative forms, specific embodiments are shown by way of example in the drawings and are described in detail herein. However, embodiments of the disclosure are not limited to the exemplary or specific forms described. Rather, the disclosure covers all modifications, equivalents, and alternatives falling within the scope of the appended claims.

The terms “invention,” “the invention,” “this invention,” “the present invention,” “the present disclosure,” or “the disclosure” as used herein are intended to refer broadly to all the subject matter disclosed in this document, the drawings or figures, and to the claims. Statements containing these terms do not limit the subject matter disclosed or the meaning or scope of the claims. Embodiments covered by this disclosure are defined by the claims and not by this summary. This summary is a high-level overview of various aspects of the disclosure and introduces some of the concepts that are further described in the Detailed Description section below. This summary is not intended to identify key, essential or required features of the claimed subject matter, nor is it intended to be used in isolation to determine the scope of the claimed subject matter. The subject matter should be understood by reference to appropriate portions of the entire specification, to any or all figures or drawings, and to each claim.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates an example of an apparatus for detecting internal tooth tissue geometries, such as enamel and dentin geometry, according to one or more embodiments described herein;

FIG. 2 illustrates an example method for detecting internal tooth tissue geometries, such as enamel and dentin geometry, according to one or more embodiments described herein;

FIG. 3 illustrates an example method for detecting enamel width, according to one or more embodiments described herein;

FIG. 4 illustrates an example method for detecting dentin geometry, according to one or more embodiments described herein;

FIG. 5 illustrates an example method for detecting dentin geometry, according to one or more embodiments described herein;

FIG. 6A and 6B illustrates an example of detecting enamel thickness, according to one or more embodiments described herein; and

FIG. 7A and 7B illustrates an example of detecting dentin geometry, according to one or more embodiments described herein.

FIG. 8A and 8B illustrates an example of detecting dentin geometry, according to one or more embodiments described herein.

FIG. 9 illustrates one example of a method of determining which images from an intraoral scan are most likely to include dentin.

Note that the same numbers are used throughout the disclosure and figures to reference like components and features.

### DETAILED DESCRIPTION

Described herein are methods and apparatuses (e.g., systems, devices, etc. including software, hardware and/or firmware) for detecting the location and geometry of internal tooth tissues, such as the enamel and dentin of a patient’s teeth from scans of a patient’s teeth. These methods and apparatuses may automatically or semi-automatically determine the location and shape of enamel and dentin of a patient’s teeth from scans of a patient’s teeth. These methods and apparatus may be used with an intraoral scanner, and in some embodiments may be included as part of the intraoral scanner. In some embodiments these methods and apparatuses may receive scan information but are not necessarily part of (or integrated with) an intraoral scanner.

Also described herein are methods and apparatuses for determining which images may best show the internal structure, such as the dentin and enamel of a patient’s teeth, from an intraoral scan by scoring the images of a scan (e.g., the near-IR images and/or visible light images) and identifying those that are most likely to include quality images of the dentin and enamel, without necessarily identifying dentin and enamel in the images. The images most likely to have quality data of the dentin and enamel may be presented to a user (e.g., displayed, saved, transmitted) and/or may be used as an input into any of these methods for determining enamel location, shape, width, and dentin shape and location.

In general, these methods and apparatuses may determine one or more features associated with a dentin and enamel, such as an outline, a center, etc. The feature (or features) may be identified from a plurality of different images from scans of the patient’s teeth, e.g., using a pattern matching agent, such as a trained machine learning agent (“trained pattern matching agent”), which may indicate possible locations, outlines, and other features of teeth, such as the enamel and dentin. These methods and apparatuses may determine shape and location of tooth features by using one or more additional techniques, including projecting a representative feature onto a 3D model of the teeth, and based on an analysis of scans of the patient’s teeth showing the representative feature from different camera angles. Thus, the methods and apparatuses described herein may dramatically eliminate false positives and false negatives.

Also described herein are methods and apparatuses for outputting identified (e.g. automatically identified) enamel and dentin shapes, widths, etc., in a manner that simplifies dental care for dental professionals.

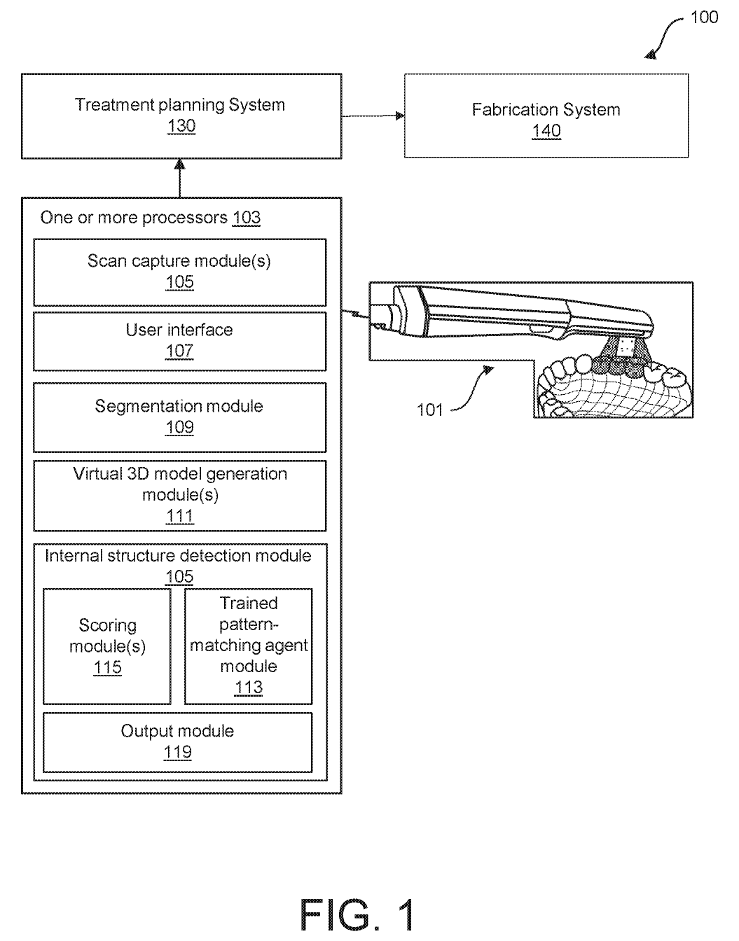

FIG. 1 schematically illustrates one example of an apparatus for determining geometries of enamel and dentin of a patient’s teeth. In this example the apparatus may include and/or may be used with an intraoral scanner. This is optional; in some examples the apparatus may be used as a stand-alone system or device (and in some examples, software) that receives scans of the patient’s teeth. Such examples may be similar the example shown in FIG. 1, but may not be directly connected or in direct electronic communication with the intraoral-scanning specific components, such as the scanner 101, scan capture module(s) 105, etc.

In the example shown in FIG. 1, the apparatus for determining geometries of enamel and dentin of a patient’s teeth may include an internal tissue detection module 105 that is functionally, and in some embodiments, electronically coupled in electronic communication and/or physically connected to the one or more processors 103 that may be part of the intraoral scanner 101. The internal tissue detection module may detect one or more types of internal tissue of the teeth, and may provide an output for display, transmission, storage, etc. The output may be graphic, such as by an annotation of virtual 3D model of the teeth including internal tissue (e.g., tissue boundaries, etc.). Alternatively or additionally the output may be textural, e.g., a written description of the internal tooth structure (tooth number and/or region of a tooth, coordinates and/or dimensions, such as thickness of the structure, mesial, distal, buccal, lingual, or occlusal position, etc.), the geometry (e.g., size, volume, 3D shape, etc.), confidence score for the identified structure type, etc. The output may summate internal tissue data from across a number of scans of the teeth and/or for each of the teeth of the patient.

Any of these apparatuses may include a user interface 107 for interacting with the internal tissue detection apparatus, and/or for displaying or otherwise outputting the detected tissues. These apparatuses may receive pre-segmented images and/or 3D models of the teeth, including the scan images and/or 3D models. In some embodiments these apparatuses may include one or more modules 109 for segmenting the images and/or the 3D model of the teeth. Alternatively or additionally, the apparatus may generate a 3D model from the scan data using one or more virtual 3D model generation modules 111.

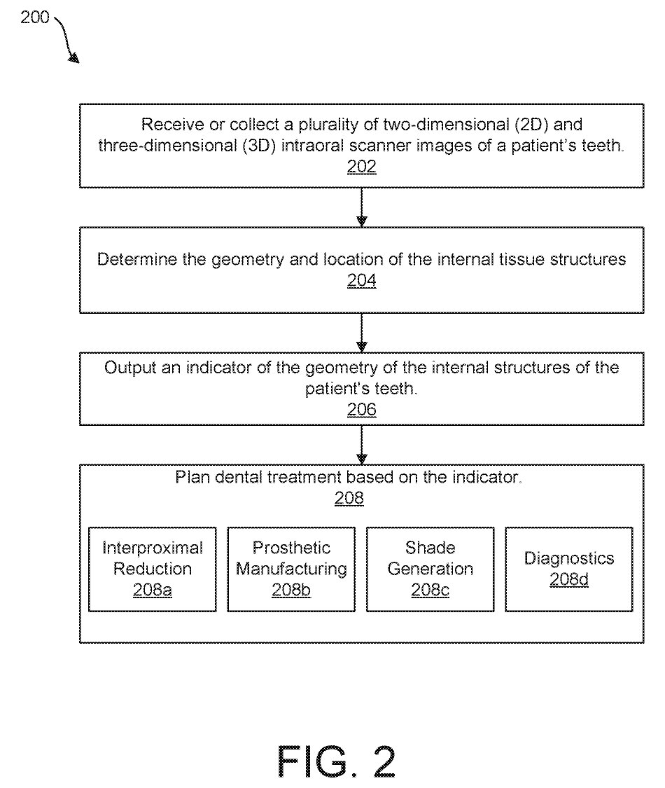

The internal tissue geometry detection module(s) 105 may include modules that may perform any of the methods described herein, as described in greater detail below, including in FIGS. 2-7. For example, in FIG. 1 the internal tissue geometry detection module 105 may include control logic for coordinating automatic detection of internal structures using scanned images having two or more scanning channels (e.g., light wavelengths, such as near-IR, red, green, blue, infrared, etc.). The detection module may be configured to operate in real time (as images are collected) and/or after images has been scanned/collected. The detection module may also coordinate input and activity of the associated modules, including one or more trained pattern matching (e.g., trained machine learning) agent modules 113. The trained pattern matching module 113 may perform any or all of the functions of the trained pattern matching agents described herein. The trained pattern matching module may receive as input one or more images in which the image(s) has multiple channels, and may output internal tissue information (e.g., location(s), width, 3D geometry, extent, etc.). The internal tissue geometry detection module(s) 105 may also include one more scoring modules 115 such as a severity scoring module and/or a confidence (or likelihood) scoring module, etc. Any of these internal tissue geometry detection modules may also include one or more internal tissue geometry consensus (or voting) modules 117. The internal tissue geometry consensus module may combine potential internal tissue geometry identified from different images having overlapping regions (e.g., overlapping regions describing the same possible internal tissue geometry from one or more of the images). The internal tissue geometry consensus modules may identify subsets of the scan images (e.g., of the images provided to the trained pattern matching module 113). In some case the internal tissue geometry consensus modules may weigh or apply weights to these images during the combining steps.

The internal tissue geometry consensus modules may also project the geometry of the internal tissues from all or some of the scan image analyzed by the trained pattern matching agent onto the virtual 3D model of the teeth. In some cases the internal tissue geometry consensus modules may include or may reference an internal tissue geometry projection module to perform these tasks.

In general, the internal tissue geometry detection module 105 may also prepare output including the detected internal tissue geometry information. The output may be configured to provide textural and/or graphic and/or 3D geometry output. In some examples the output module 119 may be configured to output to a patient file (e.g., medical and/or dental record) and/or medical/dental software or databases.

Any of the systems described herein may be used with or for treatment planning, including planning a dental, prosthodontic, and/or orthodontic treatment. For example, these apparatuses may include or connect to a treatment planning system 130 to assist a dental professional in treating a patient.

FIG. 2 illustrates one example of a method 200 for detecting internal tissue geometries. In this example, the method includes receiving or collecting a plurality of two-dimensional (2D) intraoral scanner images of a patient’s teeth at block 202. Each 2D image may include two or more channels (e.g., a near-infrared and a visible light channel). The method may also include receiving or collecting three-dimensional (3D) scan data based on the intraoral scanner images of a patient’s teeth at block 202. The 3D scan data may include a 3D digital surface model of the patient’s dentition.

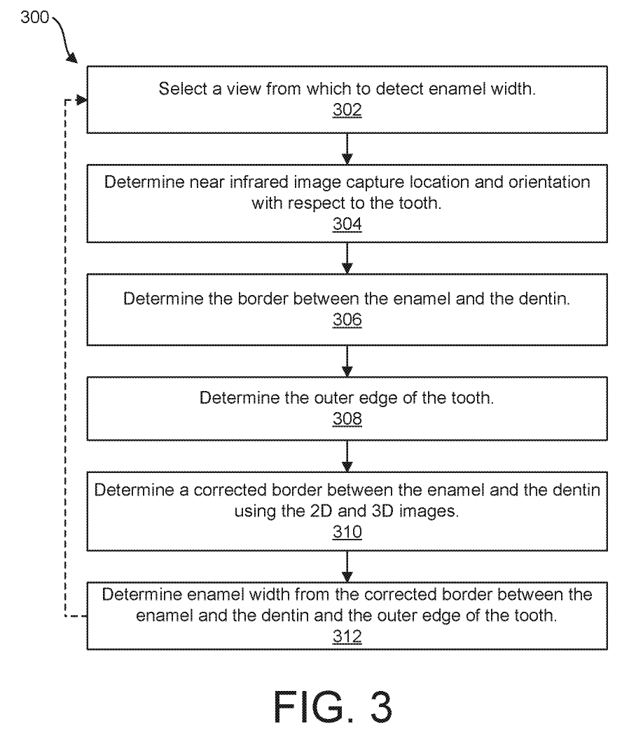

The internal tissue geometry may be identified for all or some images of the plurality of 2D intraoral scanner images and 3D intraoral scanner images at block 204. The geometry may be determined using one or more methods, such as those disclosed herein in FIGS. 3-7. Methods may include enamel thickness from single or multiple images such as method 300, dentin geometry from single or multiple images, such as method 400, dentin geometry from templates, such as method 500, and etc.

Once generated, the internal tissue geometry and/or data may be output 206, e.g., as a tissue geometry data structure, textural description of the tissue, a 3D model of the patient’s teeth, etc.

At block 208 the method 200 may plan dental treatment based on the indicator. For example, in interproximal reduction at block 208a, a mesial and distal enamel width for a tooth or teeth may be used to determine where and how much enamel is or can be safely removed from an interproximal region on one or more teeth.

For some orthodontic treatments where teeth are repositioned from an initial arrangement towards a final arrangement, it may be useful to slightly reduce the tooth width, to enable tooth movement and final tooth positioning that would not be possible without reducing the tooth width. Dentists use interproximal reduction on the mesial or distal walls of some teeth. When interproximal reduction is used between neighboring teeth, during treatment planning the dentist or treatment planning algorithm decides whether and how much enamel to remove from distal wall of a first tooth or the mesial wall of a second tooth, or from both. Knowing where the enamel is thicker can help the decide where and how much enamel to remove and optimize the decision and resulting treatment outcomes.

By automatically calculating the mesial and distal enamel width for all teeth right after scanning with near infrared and 3D surface scanning intraoral scanner, the interproximal reduction plan may be generated immediately after scanning.

Similarly, interproximal reduction decisions and planning may be based on and/or use the 3D dentin model and/or 3D enamel or surface model to avoid reductions that may compromise enamel and dentin integrity.

Interproximal reduction may be incorporated into an orthodontic treatment plan to create space for planned tooth movement, reduce crowding, improve alignment, and facilitate seating and tracking of an orthodontic appliance such as an aligner. In some embodiments, a digital model of the patient's dentition is analyzed to identify one or more interproximal locations at which enamel reduction may be performed. The treatment plan may specify, for each selected interproximal location, whether interproximal reduction is recommended, the teeth involved, the side or surface to be treated, the amount of material to be removed, and the stage of treatment at which the reduction is to occur, such as though material removal, which may be performed by a dental professional.

In some examples, interproximal reduction is planned in coordination with a series of orthodontic aligners. For example, a treatment plan may call for a first set of aligners to initiate movement of selected teeth, followed by interproximal reduction at one or more contact regions once sufficient access has been created, and then delivery of one or more subsequent aligners configured to continue movement into the space created by the reduction. The interproximal reduction may therefore be scheduled before delivery of a particular aligner, at the appointment when a particular aligner is delivered, or after a defined number of stages in the aligner sequence. In this way, the timing and amount of reduction may be selected to support predicted tooth movement while avoiding unnecessary removal of enamel.

The treatment plan may further include data indicating a maximum permissible reduction at a given interproximal location based on estimated enamel thickness, tooth anatomy, tooth position, and/or other patient-specific characteristics. In some embodiments, the plan may present a clinician with a visualization of the proposed reduction sites together with quantitative values indicating available enamel and recommended reduction amounts, thereby assisting the clinician in determining whether the prescribed interproximal reduction is appropriate. The information associated with block 208a may be used as part of diagnosis, appliance design, staging of aligner treatment, and chairside execution of the orthodontic treatment plan.

In some embodiments, an orthodontic treatment plan may be configured to move a patient's teeth from a first arrangement toward a second arrangement over a series of stages. The first arrangement may correspond to an initial arrangement of the patient's teeth, for example as represented by scan data, image data, impression data, or a digital model of the patient's dentition, such as described herein. The second arrangement may correspond to a target arrangement of the teeth, such as a desired arrangement associated with an improved alignment, occlusion, or aesthetic outcome. The treatment plan may define a plurality of intermediate arrangements between the first arrangement and the second arrangement, with each intermediate arrangement corresponding to a respective stage of treatment.

Each stage may correspond to the application of a dental appliance provided by, for example a dental professional, for application on a patient’s teeth a period of time. For example, in some embodiments using orthodontic aligners, a first provided appliance may be configured to urge one or more teeth from the first arrangement toward a first intermediate arrangement during a first wear period, a second provided appliance may be configured to urge one or more teeth from the first intermediate arrangement toward a second intermediate arrangement during a second wear period, and so on until the teeth reach, or substantially reach, the second arrangement. The period of time associated with a given stage may be predetermined, such as a number of days or weeks, and may be selected based on the magnitude and type of planned tooth movement, patient-specific considerations, and/or practitioner preference.

In some implementations, the treatment plan may specify tooth movements for individual teeth or groups of teeth at each stage. Such movements may include translation, rotation, intrusion, extrusion, tipping, torque, bodily movement, arch expansion, space closure, and/or other movements. The treatment plan may also specify one or more adjunctive procedures or events to occur at selected stages, such as attachment placement or removal, interproximal reduction, use of auxiliaries, or other clinical interventions. In some embodiments, the staged treatment plan may coordinate both appliance-driven movement and additional treatment actions to progressively move the patient's teeth from the first arrangement toward the second arrangement.

A dental appliance associated with a given stage may be designed based on a corresponding digital representation of the patient's dentition at that stage and a target arrangement for a subsequent stage. For example, a processor may generate digital models corresponding to successive tooth arrangements and may generate appliance geometry configured to apply forces to selected teeth so as to produce planned movement over the corresponding wear period. In some embodiments, a series of dental appliances may be generated, each appliance corresponding to a different stage of the treatment plan and configured to be worn in a prescribed sequence.

In some embodiments, one or more of the dental appliances may be fabricated using a fabrication machine that may be part of a fabrication system 140. For example, data representing the geometry of a dental appliance, a mold for forming the dental appliance, or a positive model of the patient's dentition may be provided to a fabrication machine, such as a three-dimensional printer, thermoforming system, milling machine, computer numerical control machine, or other manufacturing device. The data may be generated as part of a treatment plan. The fabrication machine may fabricate the dental appliance directly, or may fabricate an intermediate article, such as a mold or physical dental model, from which the dental appliance is formed. In examples involving aligners, a physical model corresponding to a stage arrangement may be fabricated and a polymer sheet may be thermoformed over the model to produce an aligner. In some embodiments, the appliance itself may be additively manufactured based on the digital appliance geometry.

The enamel and/or dentin geometry or data may also be used for prosthodontic modeling and manufacturing at block 208b, such as for use in building a restorative crown on a prepared tooth, or an implant. A restoration with visual qualities that match the neighboring natural teeth, including qualities such as color, transparency, and translucency, aids in giving the teeth and resulting smile a natural appearance.

The external shape and color, along with the internal tooth structure affects the visual perception of the tooth. Enamel and dentin geometry, transparency and opacity give the teeth its external appearance, which may change based on viewing angle. For example, anterior teeth have a thin occlusal area which may be made entirely of enamel, so when viewed at certain angles those areas are translucent.

Given an estimated shape of the dentin and enamel, from the methods discussed herein, a crown may be manufactured in multiple layers to simulate the original tooth. The methods used herein may be used to detect the enamel and dentin shapes from a pre-treatment scanning procedure, such as a scan of the tooth before preparing it for the new crown (before removing tooth material form the natural crown of the tooth), from a historical scan that contains the tooth that is to be restored, and/or the opposite corresponding tooth (same jaw, other side) that usually has very similar qualities, such as using the left upper incisor as a model for a prosthetic crown on a right upper incisor.

The dentin and/or enamel geometry or information along with the tooth shade measurements may be used by a prosthetic design module as part of the treatment planning to design a crown, bridge, veneer, or other prosthetic or implant. The treatment planning may output a multi-material 3D model of the prosthetic which may be used to fabricate, such as by 3D printing, a multiple material prosthetic, such as one using materials with different visual qualities including transparency, opacity, refractive qualities, etc., where the internal dentin shape may be fabricated by an opaque material having a shade and the external layers may be printed using a semi-transparent material having a shade. The shades may be different or similar. Such a prosthetic provides the restored tooth with an accurate approximation of the visual qualities of the original, natural tooth it replaces or restores.

In some embodiments, a restoration may be used as part of a restorative treatment and associated treatment plan for restoring a patient's tooth or teeth with a prosthetic. The treatment plan may specify that one or more teeth are to receive a restoration such as a veneer, crown, bridge, inlay, onlay, coping, implant-supported restoration, or other prosthetic. For example, the treatment plan may define a condition of a patient's tooth before treatment, a prepared condition of the tooth after reduction or other preparation, and a restored condition in which the prosthetic is received on, over, around, or adjacent to the prepared tooth. The restoration may form part of a planned progression from an initial condition of the patient's dentition to a treated condition associated with improved function, anatomy, occlusion, and/or aesthetics.

In some embodiments, the treatment plan may specify one or more restorative procedures associated with placement of the restoration. For example, the treatment plan may include preparation of a tooth to receive the restoration, determination of a margin or finish line, evaluation of available restorative space, design of the restoration, placement of a temporary restoration, seating of a definitive restoration, and/or evaluation or adjustment of contacts and occlusion.

In some embodiments, a digital model of the patient's dentition may be used to generate a model of the prepared tooth and a corresponding model of the restoration to be received by the prepared tooth. The tooth or teeth may be prepared by automated means, such as the CNC of a material removal machine or manually by a dental professional. The treatment plan may associate the restoration with one or more characteristics such as contour, thickness, shade, fit, emergence profile, occlusal anatomy, proximal contact, or margin geometry. The treatment plan may further specify whether the restoration is to be bonded, cemented, affixed to an abutment, or otherwise secured in place. In this manner, the restoration may be planned as part of a restorative treatment intended to return the patient's tooth or teeth to a desired functional and/or aesthetic state.

The information associated with block 208b may be used in diagnosis, restorative treatment planning, prosthetic design, tooth preparation guidance, and seating or verification of the restoration. In some embodiments, the treatment plan may also coordinate the restoration with one or more fabrication-related steps, such as generating data for manufacture of the restoration, such as the prosthetic an intermediate article associated with the restoration, such as a mold or wax model for use in making a mold that is subsequently used for fabrication of the prosthetic.

In some embodiments, a restorative treatment plan may be used to prepare a patient's tooth or teeth to receive a dental prosthetic. The restorative treatment plan may relate to a single tooth or multiple teeth and may be used in connection with a veneer, crown, bridge, inlay, onlay, coping, implant-supported restoration, or other dental prosthetic. A first digital model may represent an initial arrangement or condition of the patient's dentition, including one or more teeth before preparation. The restorative treatment plan may define a prepared state for one or more teeth, such as a state in which tooth structure has been reduced, contoured, reshaped, and/or otherwise modified to receive the dental prosthetic. The prepared state may correspond to a target preparation geometry for a prepared tooth.

In some embodiments, the restorative treatment plan may specify one or more preparation parameters for the tooth or teeth to be treated. Such parameters may include, for example, reduction amounts at selected regions of a tooth, locations of finish lines or margins, taper, occlusal or incisal clearance, axial reduction, facial or lingual reduction, proximal reduction, path of insertion considerations, and/or other features associated with preparing a tooth for a prosthetic restoration based on the internal and external structures of the teeth. The restorative treatment plan may therefore assist a practitioner in determining how the patient's tooth or teeth are to be modified to receive the prosthetic.

In some embodiments, the restorative treatment plan may generate a model of the prepared tooth and a model of the dental prosthetic to be received by the prepared tooth. For example, a processor may generate a digital model of the prepared tooth based on scan data, image data, one or more preparation parameters, and/or a simulated preparation of an initial tooth model, as discussed herein. The processor may also generate a digital model of the dental prosthetic configured to fit on, over, around, or adjacent to the prepared tooth. In the case of a crown, the model of the prosthetic may define an interior surface configured to mate with the prepared tooth and an exterior surface configured to restore anatomy, function, and/or appearance. In the case of a veneer, the model may define a thin shell configured to bond to a prepared facial surface of a tooth. In the case of a bridge, the model may define one or more retainer portions and one or more pontic portions configured to restore a missing tooth space.

In some implementations, the model of the prosthetic may be generated in coordination with the model of the prepared tooth such that the prosthetic has a desired fit, thickness, contour, occlusion, contact relationship, emergence profile, and/or aesthetic characteristic. The restorative treatment plan may further include determining or presenting a margin line, evaluating clearance, evaluating space available for restorative material, evaluating contact with adjacent or opposing teeth, and/or revising the preparation model or prosthetic model based on one or more such evaluations. Thus, the restorative treatment plan may be used to define both the geometry of the prepared tooth and the geometry of the prosthetic restoration intended to be received by the prepared tooth.

In some embodiments, one or more models associated with the restorative treatment plan may be used in fabricating a dental prosthetic using a fabrication machine. For example, data representing the prosthetic model, the prepared tooth model, a die model, or a mold associated with the prosthetic may be provided to a fabrication machine as part of the fabrication system 140, such as a milling machine, computer numerical control machine, three-dimensional printer, or other manufacturing device. The fabrication machine may fabricate the dental prosthetic directly from restorative material, or may fabricate an intermediate article, such as a mold, die, wax pattern, coping, framework, or other component from which the prosthetic is further processed or formed. In some examples, a crown, veneer, bridge component, or other prosthetic may be milled from a ceramic, composite, metal, polymer, or other dental material based on the digital prosthetic model. In other examples, the prosthetic or an intermediate article may be additively manufactured based on the digital model.

At block 208c, shade generation, such as for use in prosthetic manufacturing, filings, and other restorative work, may be accomplished during treatment planning by using traditional shade capture data, such as by capture and comparison of physical shade guides along with knowledge of the internal tissue structures of the tooth for which shade is desired. For example, the captured shade, along with the determined internal tissue geometry can, through ray tracing and other methods, be updated to a corrected shade for use in fabricating restorative objects and materials, such as crowns, bridges, veneers, filings, etc. For example, shade guides may be captured from multiple angles while placed near teeth in the mouth. Ray tracing from each of the multiple angles may be used to correct the captured shade of the teeth based on the shade guide and internal structures of the teeth.

At block 208d, enamel and/or dentin geometry can be used with other data for aiding the dentist’s analysis of patient dentition health and treatment planning. For example, a patient’s teeth may be scanned over time allowing for longitudinal analysis, such as analysis over time, wherein the thickness of the patient’s enamel may be measured and tracked over time. Tooth wear may cause the enamel to thin. If decreasing enamel thickness is detected over time, the scanner or dental system may provide feedback, such as a warning, to a dental professional indicating that the enamel is wearing. The system may provide an estimate of how long before the patient’s should be treated, such as through a restorative procedure, to protect the dentin.

If thin enamel is detected, such as based on enamel width and dentin position, even without wear, such as a natural biologically thin enamel on anterior teeth, which are more susceptible to breakage, the system may highlight the thin enamel to the dentist and patient, to provide treatment planning and decisions as to the use of a veneer (or other treatment), which may be beneficial to the patient.

As mentioned, any of the methods or portions of these methods may be performed by one or more modules including executable instructions. Modules may also be referred to herein as engines. A module may be implemented as an engine, as part of an engine or through multiple engines. As used herein, an engine includes one or more processors or a portion thereof. A portion of one or more processors can include some portion of hardware less than all of the hardware comprising any given one or more processors, such as a subset of registers, the portion of the processor dedicated to one or more threads of a multi-threaded processor, a time slice during which the processor is wholly or partially dedicated to carrying out part of the engine’s functionality, or the like. As such, a first engine and a second engine can have one or more dedicated processors, or a first engine and a second engine can share one or more processors with one another or other engines. Depending upon implementation-specific or other considerations, an engine can be centralized, or its functionality distributed. An engine can include hardware, firmware, or software embodied in a computer-readable medium for execution by the processor. The processor transforms data into new data using implemented data structures and methods, such as is described with reference to the figures herein.

The modules described herein, and/or the engines, through which the systems and devices described herein can be implemented, can be cloud-based or local. As used herein, a cloud-based module or engine may run applications and/or functionalities using a cloud-based computing system. All or portions of the applications and/or functionalities can be distributed across multiple computing devices, and need not be restricted to only one computing device. In some embodiments, the cloud-based modules or engines can execute functionalities and/or modules that end users access through a web browser or container application without having the functionalities and/or modules installed locally on the end-users’ computing devices.

As used herein, datastores are intended to include repositories having any applicable organization of data, including tables, comma-separated values (CSV) files, traditional databases (e.g., SQL), or other applicable known or convenient organizational formats. Data stores can be implemented, for example, as software embodied in a physical computer-readable medium on a specific-purpose machine, in firmware, in hardware, in a combination thereof, or in an applicable known or convenient device or system. Datastore-associated components, such as database interfaces, can be considered "part of" a datastore, part of some other system component, or a combination thereof, though the physical location and other characteristics of datastore-associated components is not critical for an understanding of the techniques described herein.

Data stores can include data structures. As used herein, a data structure is associated with a particular way of storing and organizing data in a computer so that it can be used efficiently within a given context. Data structures are generally based on the ability of a computer to fetch and store data at any place in its memory, specified by an address, a bit string that can be itself stored in memory and manipulated by the program. Thus, some data structures are based on computing the addresses of data items with arithmetic operations; while other data structures are based on storing addresses of data items within the structure itself. Many data structures use both principles, sometimes combined in non-trivial ways. The implementation of a data structure usually entails writing a set of procedures that create and manipulate instances of that structure. The datastores described herein can be cloud-based datastores. A cloud-based datastore is a datastore that is compatible with cloud-based computing systems and engines.

The automated agents described herein may implement one or more procedures based on 3D virtual representations of teeth taken from subjects. A “tooth type,” as used herein, may refer to a specific tooth in the mouth of a human being. A tooth type may include any specific tooth identified according to an “anatomical tooth identifier,” which as used herein, may refer to any identifier used to anatomically identify the tooth type. Examples of anatomical tooth identifiers include identifiers of a universal or other tooth numbering system, character identifiers, image(s), etc. A “virtual 3D” model or representation may refer to a 3D rendering of a tooth or teeth. Examples of virtual 3D models include animated 3D renderings, composite 3D renderings assembled from 2D images, etc. A 3D virtual representation may have one or more “virtual surface contours,” or contours that define surfaces of the tooth in a virtual 3D space.

In various implementations, an automated tooth modeling engine(s) may implement one or more automated agents configured to describe 3D virtual representations of teeth or tooth features with mathematical 3D descriptors that use spatial parameters. A mathematical 3D descriptor, as used herein, may refer to a mathematical function that represents virtual surface contours and/or other portions of 3D virtual representations of teeth according to spatial parameters. Examples of mathematical 3D descriptors include Elliptical Fourier Descriptors (EFDs), spherical harmonic functions that use voxelized spheres, and spherical harmonic functions that use non-voxelized spheres. A spatial parameter may refer to a parameter that relates to a spatial element. Examples of spatial parameters include coordinates, e.g., locational coordinates, identified along orthogonal systems, such as three translational planes, 3D polar coordinates, etc. As noted herein, mathematical 3D descriptors may parametrically represent a 3D virtual representation, or represent that 3D virtual representation according to one or more parameters, such as spatial parameters.

In various implementations, mathematical 3D descriptors may form a “mathematical 3D descriptor space,” or a datastore of mathematical 3D descriptors with descriptor locations assigned for each mathematical 3D descriptor space. “Descriptor locations,” as used herein, may refer to unique coordinates in the mathematical 3D descriptor space where each mathematical 3D descriptor reside. In various implementations, descriptor locations may be used to define “descriptor distances,” or differences in distances between descriptor locations of mathematical 3D descriptors in a mathematical 3D descriptor space.

FIG. 3 depicts a method 300 for detecting enamel width. The method 300 for detecting enamel width may detect enamel width from one or more 2D near infrared images of the internal structure of the patient’s teeth and 3D surface data of the patient’s teeth. Method 300 may take place as part of method 200, such as at block 204.

At block 302 a tooth and a view from which to detect enamel width may be determined. For example, the view may be an occlusal view taken perpendicular to the occlusal surface or axis of a tooth, a buccal view, taken perpendicular to the mesial surface or axis of the patient’s tooth, a lingual view taken perpendicular to the lingual surface or axis of the tooth, or another view. In some embodiments, the selected view may be a view perpendicular to an enamel region of interest. For example, for interproximal enamel width in a molar tooth, the occlusal direction can be preferred.

At block 304, a near infrared image captured of the tooth from the selected view is retrieved or selected from the 2D near infrared images captured by the intraoral scanner. The image may be retrieved or selected based on the 3D image data. For example, an intraoral scanner may capture 2D near infrared and 3D data at the same time or in close time proximity to each other. The position and orientation of the scanner with respect to the teeth may be determined based on the 3D image data captured during the 2D and 3D scanning process. For example, the position of the scanner may be determined based on the captured 3D data points in a frame of a 3D scan. The 3D scan data may be evaluated to determine at which point in time the scanner was most closely or sufficiently close to the position and/or orientation selected in block 302. The near infrared image from the scanner captured closest in time, immediately before, or immediately after the 3D data that is closes to the desired view, may be selected for use in determining the enamel width.

When the intraoral scanner captures a 2D image and a corresponding 3D scan frame at the same time, or within a sufficiently short time interval, the 2D image may be associated with that 3D scan frame. The 3D scan frame includes a set of 3D points representing a portion of the patient’s dentition. That set of 3D points may be registered to the 3D model of the patient’s teeth by comparing the captured 3D points to corresponding surface geometry of the 3D model. For example, the captured 3D points may be aligned to the 3D model using one or more rigid registration techniques, surface matching techniques, feature-based matching techniques, or iterative closest point techniques. Once the captured 3D points are aligned to the 3D model, the pose of the scanner at the time the 3D scan frame was captured may be determined relative to the 3D model. Because the 2D image was captured at the same time, or within a threshold time of that 3D scan frame, the pose of the scanner for the 2D image may be taken to be the same as, or derived from, the pose determined for the 3D scan frame. In this manner, the location and orientation of the 2D image relative to the patient’s teeth and relative to the 3D model may be determined.

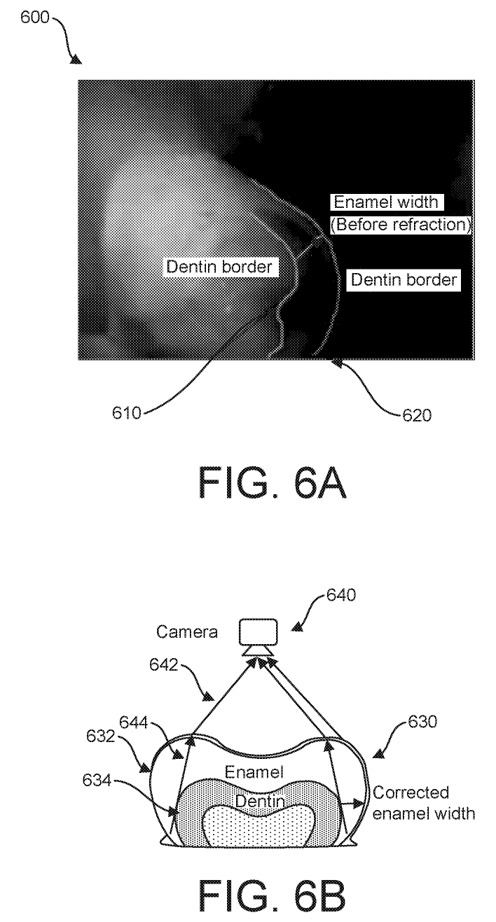

At block 306 a candidate border between the enamel of the dentin in the near infrared image may be detected. FIG. 6A depicts a near infrared image 600 of tooth from an occlusal view, though the methods described herein apply to other views of teeth. In some embodiments, the candidate border may correspond to a transition in pixel intensity, texture, scattering pattern, translucency, or other image characteristic associated with a transition between enamel and dentin. In some embodiments, the image may first be preprocessed before identifying the candidate border, such as by normalization, denoising, contrast enhancement, deblurring, glare suppression, thresholding, filtering, registration to another image channel, or other preprocessing operations. In some embodiments, identifying the candidate border may include identifying one or more candidate pixels, edges, contours, boundary segments, masks, or regions corresponding to dentin and/or enamel, from which a border may be inferred. In some embodiments, the candidate border may be identified directly from the near infrared image, while in other embodiments the candidate border may be identified using the near infrared image together with one or more additional image channels, such as visible-light image data, fluorescence data, or data derived from the 3D scan. In some embodiments, multiple candidate borders may initially be identified, and one or more of the candidate borders may be retained for further processing based on a confidence score, anatomical plausibility, agreement with neighboring images, or agreement with the 3D model of the tooth.

At block 308, using algorithms such as edge detection with or without other algorithms, such as contrast enhancement, sharpening, etc., a line 610 that represents the border between the enamel and the dentin in the desired region is generated. Similar methods, such as edge detection, with or without other algorithms, such as contrast enhancement, sharpening, etc., may be used to detect the contour 620 of the outer edge of the tooth in the near infrared image. In some embodiments, the near infrared image may be used in combination with the 3D model geometry from the intraoral scan to detect the contour 620 of the tooth edge, such as the outer surface of the tooth. For example, the near infrared image may be used in combination with the 3D model geometry from the intraoral scan to detect the contour 620 of the tooth edge, such as the outer surface of the tooth, by determining a pose of the camera relative to the 3D model at the time the near infrared image was captured and projecting the outer surface of the 3D model into the image plane of the near infrared image. The projected outer surface may define an expected location of the tooth edge in the near infrared image. One or more image-processing algorithms, such as edge detection, thresholding, gradient analysis, contour fitting, or machine-learning-based segmentation, may then be applied to identify candidate contours in the near infrared image, and a candidate contour that matches or is sufficiently close to the projected outer surface may be selected as contour 620. In some embodiments, the projected outer surface may define a search region within which the contour 620 is detected, thereby reducing false detections caused by noise, reflections, neighboring teeth, or internal tooth features

In some embodiments, the detection of the edges of the dentin and the enamel, such as the border between the dentin and the enamel and also the outer surface of the enamel as discussed in blocks 306 and 308 may be carried out using a trained pattern matching agent such as a trained artificial intelligence algorithms, such as trained machine learning and/or neural networks.

At block 310 a corrected border between the enamel and the dentin using the 2D and 3D images is generated. With reference to FIG. 6B, the 3D surface model, the detected edges and/or area of the dentin and/or enamel may be used to determine the enamel width. For example, the position of the camera 630 relative to the tooth model 630 when the near infrared image was captured may be modeled. The areas and/or edges of the dentin 632 and/or enamel 634 determined at blocks 304 and/or 306 may also be positioned within the 3D model of the tooth crown 630. Based on the modeled positions of the 3D surface model of the tooth, the areas and/or edges of the dentin and the enamel, the camera model, and the enamel refraction index, the position of the enamel and/or dentin edges and/or areas may be corrected. For example, a ray tracing simulation may simulate light rays passing between and through the dentin, enamel, and the air from the tooth to the camera sensor to correct the position and geometry of the areas and/or positions of the dentin and/or enamel within the tooth.

When near infrared light carrying information about internal tooth structure travels from a location in or near the dentin, through the enamel, and then into the air toward the camera, the path of that light may be altered by differences in optical properties between the dentin, enamel, and air. For example, the light may refract at the dentin-enamel boundary and again at the enamel-air boundary because those materials may have different refractive indices. The light may also scatter within the enamel or dentin. As a result, a feature visible in the near infrared image, such as an apparent dentin-enamel border, may appear shifted, distorted, enlarged, reduced, or otherwise displaced relative to its true position within the tooth.

A ray tracing simulation may model this process by defining one or more candidate internal structures within the tooth and then simulating rays that travel from those candidate structures toward the camera through the tooth materials. In some embodiments, the simulation may use the known or estimated outer tooth geometry from the 3D model, one or more estimated internal boundaries, and one or more optical properties of enamel and dentin, such as refractive index, scattering coefficient, absorption coefficient, or combinations thereof. The simulation may then determine how light rays originating from or reflected by the dentin and/or enamel would propagate through the tooth and where those rays would intersect the image plane or camera sensor.

The simulated image positions of those rays may then be compared to the actual locations of features observed in the near infrared image. If the simulated projection of an internal boundary does not match the observed boundary in the near infrared image, the system may iteratively adjust the estimated position, curvature, thickness, or shape of the internal structure until the simulated image better matches the observed image. In this way, the ray tracing simulation may be used to back-calculate or infer the true position and geometry of the dentin, enamel, or dentin-enamel boundary from the optically distorted appearance of those structures in the captured near infrared image.

At block 312 the enamel width may be measured based on the corrected areas and/or edges between the enamel-dentin contour and the outer surface of the tooth. The measurement may be in a direction perpendicular to the outer surface of the tooth or parallel to the mesial-distal, buccal-lingual, or root-coronal axis of the tooth.

The process may be repeated for the same tooth from different perspectives, for each of the selected views. In some embodiments, the enamel width may be calculated for each 2D image captured by the intraoral scanner, or from a plurality of images from the intraoral scanner. For example, a first image or images may be evaluated from an occlusal direction to detect mesial, distal, buccal, and/or lingual enamel thickness. Second and more images may evaluate the tooth from a buccal or lingual view to determine enamel thickness in the occlusal, mesial, and distal portions of the teeth.

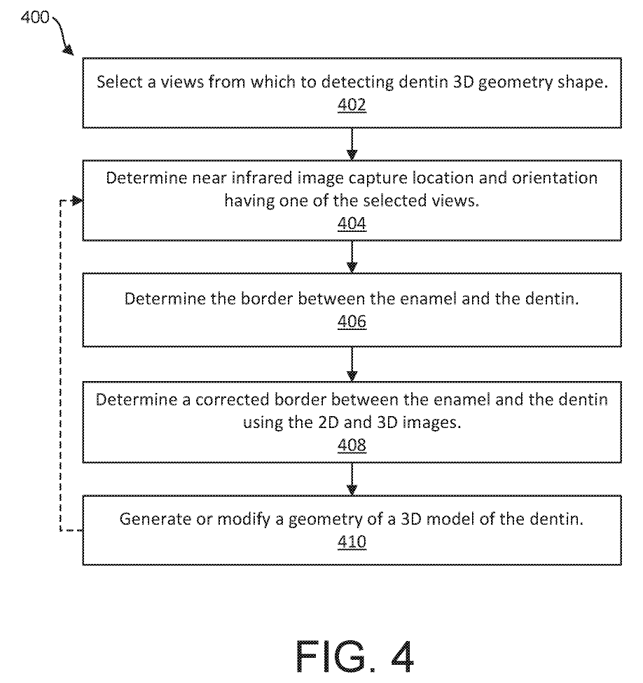

FIG. 4 depicts a method 400 for detecting dentin location and geometry. The method 300 for detecting dentin location and geometry may detect dentin location and geometry from one or more 2D near infrared images of the internal structure of the patient’s teeth and 3D surface data of the patient’s teeth. Method 400 may take place as part of method 200, such as at block 204.

At block 402 a tooth and a view from which to detect dentin location and geometry may be determined. For example, the view may be an occlusal view taken perpendicular to the occlusal surface or axis of a tooth, a buccal view, taken perpendicular to the mesial surface or axis of the patient’s tooth, a lingual view taken perpendicular to the lingual surface or axis of the tooth, or another view. In some embodiments, the selected view may be a view perpendicular to an dentin region of interest.

At block 404, a near infrared image captured of the tooth from the selected view or views is retrieved or selected from the 2D near infrared images captured by the intraoral scanner. The image may be retrieved or selected based on the 3D image data. For example, an intraoral scanner may capture 2D near infrared and 3D data at the same time or in close time proximity to each other. The position and orientation of the scanner with respect to the teeth may be determined based on the 3D image data captured during the 2D and 3D scanning process. For example, the position of the scanner may be determined based on the captured 3D data points in a frame of a 3D scan. The 3D scan data may be evaluated to determine at which point in time the scanner was most closely or sufficiently close to the position and/or orientation selected in block 402. The near infrared image from the scanner captured closest in time, immediately before, or immediately after the 3D data that is closest to the desired view, may be selected for use in determining the enamel width.

When the intraoral scanner captures a 2D image and a corresponding 3D scan frame at the same time, or within a sufficiently short time interval, the 2D image may be associated with that 3D scan frame. The 3D scan frame includes a set of 3D points representing a portion of the patient’s dentition. That set of 3D points may be registered to the 3D model of the patient’s teeth by comparing the captured 3D points to corresponding surface geometry of the 3D model. For example, the captured 3D points may be aligned to the 3D model using one or more rigid registration techniques, surface matching techniques, feature-based matching techniques, or iterative closest point techniques. Once the captured 3D points are aligned to the 3D model, the pose of the scanner at the time the 3D scan frame was captured may be determined relative to the 3D model. Because the 2D image was captured at the same time, or within a threshold time of that 3D scan frame, the pose of the scanner for the 2D image may be taken to be the same as, or derived from, the pose determined for the 3D scan frame. In this manner, the location and orientation of the 2D image relative to the patient’s teeth and relative to the 3D model may be determined.

At block 406 a border between the enamel of the dentin in the near infrared image may be detected. FIG. 6A depicts a near infrared image 600 of tooth from an occlusal view, though the methods described herein apply to other views of teeth.

The detection may include using algorithms such as edge detection with or without other algorithms, such as contrast enhancement, sharpening, etc., a line 610 that represents the border between the enamel and the dentin in the desired region is detected.

In some embodiments, the candidate border may correspond to a transition in pixel intensity, texture, scattering pattern, translucency, or other image characteristic associated with a transition between enamel and dentin. In some embodiments, the image may first be preprocessed before identifying the candidate border, such as by normalization, denoising, contrast enhancement, deblurring, glare suppression, thresholding, filtering, registration to another image channel, or other preprocessing operations. In some embodiments, identifying the candidate border may include identifying one or more candidate pixels, edges, contours, boundary segments, masks, or regions corresponding to dentin and/or enamel, from which a border may be inferred. In some embodiments, the candidate border may be identified directly from the near infrared image, while in other embodiments the candidate border may be identified using the near infrared image together with one or more additional image channels, such as visible-light image data, fluorescence data, or data derived from the 3D scan. In some embodiments, multiple candidate borders may initially be identified, and one or more of the candidate borders may be retained for further processing based on a confidence score, anatomical plausibility, agreement with neighboring images, or agreement with the 3D model of the tooth.

In some embodiments, the detection of the edges of the dentin and the enamel, such as the border between the dentin and the enamel and may be carried out using a trained pattern matching agent such as a trained artificial intelligence algorithms, such as trained machine learning and/or neural networks.

At block 408 a corrected border between the enamel and the dentin using the 2D and 3D images. With reference to FIG. 6B, the 3D surface model, the detected edges and/or area of the dentin and/or enamel may be used to determine the enamel width. For example, the position of the camera 630 relative to the tooth model 630 when the near infrared image was captured may be modeled. The areas and/or edges of the dentin 632 and/or enamel 634 determined at blocks 304 and/or 306 may also be positioned within the 3D model of the tooth crown 630. Based on the modeled positions of the 3D surface model of the tooth, the areas and/or edges of the dentin and the enamel, the camera model, and the enamel refraction index, the position of the enamel and/or dentin edges and/or areas may be corrected. For example, a ray tracing simulation may simulate light rays 642, 644 passing between and through the dentin, enamel, and the air from the tooth to the camera sensor to correct the position and geometry of the areas and/or positions of the dentin and/or enamel within the tooth.

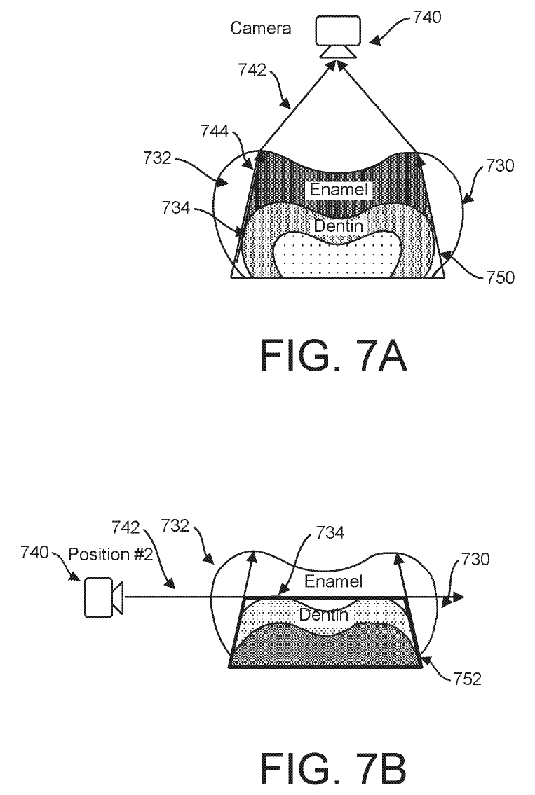

At block 410 a dentin geometry 750 may be detected based on the corrected areas and/or edges between the enamel-dentin contour. The initial geometry depicted in FIG. 7A may be based on the projection of the dentin outline within the 3D model of the tooth 730, such as by tracing rays 742, 744 through the air and the tooth. The initial 3D model of the dentin may be the occlusal surface of the tooth with side surfaces extending into the tooth and bounded or defined by the rays projecting from the camera towards to the dentin, and extending past the detected outer edge of the dentin.

The process may be repeated for the same tooth from different perspectives, for each of the selected views. For example, a second camera position, or view may be generated from a buccal/lingual perspective. An updated geometry, depicted in FIG. 7B may be generated using the process described above. At block 410, the ray tracing traces a ray 742 along an edge 734 of the dentin. The edge 734 of FIG. 7B may depict an occlusal boundary of the dentin. By applying the projection of the ray or rays 743 to the initial 3D model 750 of the dentin, an updated 3D model 752 of the dentin may be generated, a portion of the shape of which is indicated by the bold lines.

This process can be repeated by analyzing the regions of projection of the dentin from multiple angles and positions and comparing them across multiple viewpoints, the algorithm can systematically remove non-object regions from the 3D geometric shape of the dentin 750, 752. This iterative refinement process results in an increasingly accurate 3D representation of the dentin’s geometry at each iteration.

Each captured image is processed to extract dentin boundaries. Such as by using edge detection, thresholding, or machine learning-based segmentation techniques discussed herein. The extracted areas and/or edges are projected into the 3D space according to the known light source positions and camera viewpoints. Any part of the initial volume that falls outside the projected volume is marked as non-dentin removed from the 3D geometry of the dentin. The process is repeated for multiple camera positions, with each iteration further refining the estimated 3D shape.

As more viewpoints and shadow projections are analyzed, the 3D shape is further constrained. By intersecting volumes from different camera angles, an increasingly precise representation of the object's surface is obtained. Any volumetric elements that contradict multiple shadow constraints are eliminated.

The refined volumetric model may contain surface irregularities due to noise or incomplete shadow data. Surface smoothing techniques, such as mesh reconstruction, point-cloud fitting, or implicit surface modeling, may be applied to enhance model fidelity. Additional texture mapping or photometric refinement can be applied to improve the realism of the reconstructed model. Such as by applying near infrared image data to the dentin model.

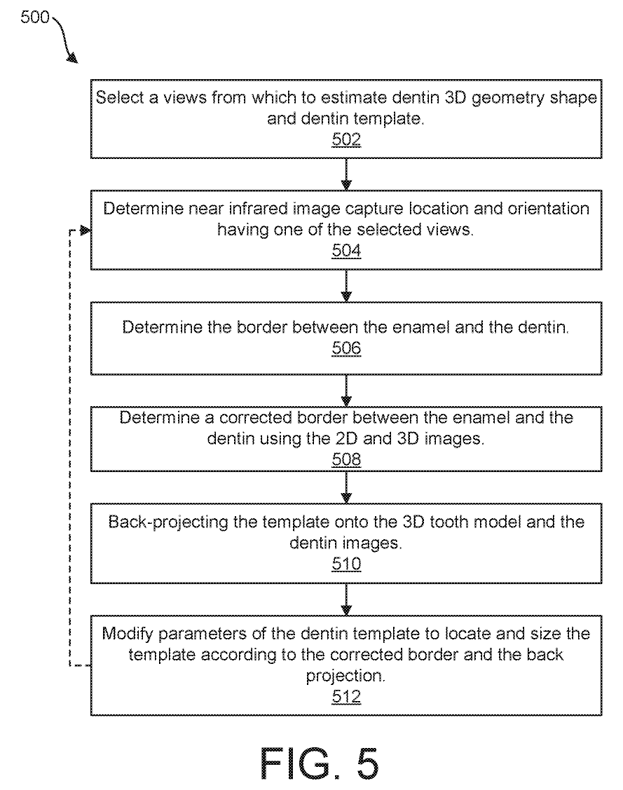

FIG. 5 depicts a method 500 of detecting a 3D geometry of a dentin using near infrared imaging and dentin templates. The method 500 for detecting dentin location and geometry may detect dentin location and geometry using near infrared images and dentin templates may use one or more 2D near infrared images of the internal structure of the patient’s teeth and 3D surface data of the patient’s teeth. Method 500 may take place as part of method 200, such as at block 204.

At block 502 a tooth and a view from which to detect dentin location and geometry may be determined along with a dentin template. For example, the view may be an occlusal view taken perpendicular to the occlusal surface or axis of a tooth, a buccal view, taken perpendicular to the mesial surface or axis of the patient’s tooth, a lingual view taken perpendicular to the lingual surface or axis of the tooth, or another view. In some embodiments, the selected view may be a view perpendicular to a dentin region of interest. The template may be a parameterized template. The template may also be a template based on a generic dentin shape for a particular tooth, such as a generic template shape for a left central incisor, a right lower canine, etc. In some embodiments, the template may be based on the 3D scan data of the tooth. Dentin often takes on a shape similar to that of the external shape of the crown of the tooth. A template based on the 3D model of the tooth, may be an adequate representation of the dentin within the tooth, when scaled, as discussed herein.

In some embodiments, a parameterized template may be a deformable three-dimensional dentin model associated with a selected tooth type and controlled by a plurality of parameters corresponding to anatomical features of the dentin. For example, for a molar tooth, the parameters may include one or more of: mesial-distal dentin width, buccal-lingual dentin width, occlusal-cervical dentin height, dentin horn height, dentin horn spacing, central fossa depth, cervical constriction, axial taper, sectional curvature, pulp chamber position, pulp chamber size, root trunk width, and root divergence. For an incisor, the parameters may include one or more of mesial-distal width, labial-lingual width, incisal-cervical height, incisal dentin thickness, cervical narrowing, lingual fossa depth, mesial curvature, distal curvature, and pulp chamber position. The values of the parameters may be adjusted to deform the template so that the template corresponds to the external 3D scan data of the patient’s tooth and to dentin boundary information detected in one or more images. In some embodiments, fitting the parameterized template may include scaling, translating, rotating, bending, tapering, expanding, contracting, or otherwise deforming the template within anatomically plausible limits for the selected tooth type.

In some embodiments, the dentin template may be a parameterized template having a default geometry for a selected tooth type and a set of adjustable parameters that define the shape and position of the dentin within the tooth. For example, the parameterized template may include parameters for overall scale, mesial-distal width, buccal-lingual width, occlusal-cervical height, cervical narrowing, dentin horn height, dentin horn spacing, sectional curvature, and pulp chamber position. The parameterized template may be initialized based on a generic dentin shape for the selected tooth type, such as a left central incisor, a right lower canine, or a first molar, and may then be deformed based on the 3D scan data of the patient’s tooth. Because dentin often takes on a shape similar to that of the external shape of the crown of the tooth, the parameterized template may in some embodiments be generated by scaling inward from the external 3D model of the tooth and then adjusting one or more of the parameters to better fit dentin boundary information detected in one or more selected views.

For example, for a maxillary central incisor, the parameterized dentin template may begin as a generic incisor dentin volume and may be adjusted using parameters corresponding to crown height, mesial-distal width, labial-lingual thickness, incisal dentin prominence, cervical taper, and pulp chamber location so that the template fits within the external 3D model of the scanned tooth and corresponds to dentin boundaries detected in one or more near infrared images.

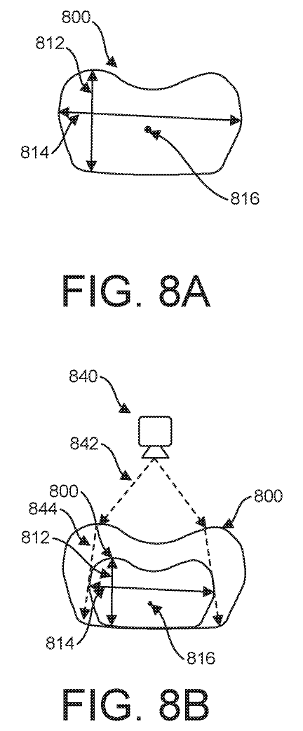

With reference to FIG. 8A, a dentin template 800 is depicted. The parameters for a dentin template may be height 812 along or parallel to the root-occlusal axis, width 814 along or parallel to the mesial-distal axis, width (or depth) along or parallel to the buccal-lingual axis of the crown, and three-dimensional (x, y, z) coordinates of a center location 816 of the dentin. In some embodiments, for example, when the template is based on the 3D scan data of the external surface of the tooth, the parameters may include a center and a scale applied to the shape.

At block 504, a near infrared image captured of the tooth from the selected view or views is retrieved or selected from the 2D near infrared images captured by the intraoral scanner. The image may be retrieved or selected based on the 3D image data. For example, an intraoral scanner may capture 2D near infrared and 3D data at the same time or in close time proximity to each other. The position and orientation of the scanner with respect to the teeth may be determined based on the 3D image data captured during the 2D and 3D scanning process. For example, the position of the scanner may be determined based on the captured 3D data points in a frame of a 3D scan. The 3D scan data may be evaluated to determine at which point in time the scanner was most closely or sufficiently close to the position and/or orientation selected in block 502. The near infrared image from the scanner captured closest in time, immediately before, or immediately after the 3D data that is closest to the desired view, may be selected for use in determining the enamel width.

At block 506 a border between the enamel and the dentin in the near infrared image may be detected, as discussed herein, such as with respect to FIGS. 2 and/or 3. FIG. 6A depicts a near infrared image 600 of tooth from an occlusal view, though the methods described herein apply to other views of teeth.

At block 508 a corrected border between the enamel and the dentin using the 2D and 3D images may be determined. With reference to FIG. 6B, the 3D surface model, the detected edges and/or area of the dentin and/or enamel may be used to determine the enamel width. For example, the position of the camera 630 relative to the tooth model 630 when the near infrared image was captured may be modeled. The areas and/or edges of the dentin 632 and/or enamel 634 determined at blocks 304 and/or 306 may also be positioned within the 3D model of the tooth crown 630. Based on the modeled positions of the 3D surface model of the tooth, the areas and/or edges of the dentin and the enamel, the camera model, and the enamel refraction index, the position of the enamel and/or dentin edges and/or areas may be corrected. For example, a ray tracing simulation may simulate light rays 642, 644 passing between and through the dentin, enamel, and the air from the tooth to the camera sensor to correct the position and geometry of the areas and/or positions of the dentin and/or enamel within the tooth.

At block 510 parameters of the dentin template may be modified. For example, the center location of the dentin may be modified to place the center of the dentin template at the location of the center of the dentin in the near infrared image aligned with the 3D model of the tooth. For example, via a projection of the 2D image on the 3D model or a projection of the 3D model on the 2D image. Similarly, the width, depth, and height parameters may also be adjusted to match projection of the template with the near infrared image of the tooth. For example, with reference to FIG. 8B, a camera 840 may be placed at the capture location of the imaging device relative to the tooth model 800 when the near infrared image was captured.

Rays 842, 844 may be traced to generate a forward or reverse projection of the template that matches the near infrared image data and at block 512, parameters may be modified based on the projection. For example, the parameters of the template may be iteratively modified until the forward or reverse projection of the template matches the near infrared image. In some embodiments, the 3D model, which may include the external surface of the tooth and a template of the dentin, may be projected on or with respect to the 2D image, or vise-versa. Then, from the relationship of the dentin on the 2D image with respect to the 3D model, the measured border from the image and the projected from the 3D model and template, and then determine whether or not they overlap. If not, the parameters of the template are updated and the 2D image and the 3D model with the updated parameters is projected again until the border or borders on the 2D image aligns with the border or borders on 3D model. In some embodiments, a subset of parameters may be modified based on a first near infrared image from a first view and a second subset of parameters may be modified based on a second near infrared image from a second view. In FIG. 8A, the parameters that define the dentin shape in a plane parallel to the root-occlusal axis and the mesial-distal axis may be modified. These parameters may include the mesial-distal width 814, the root-occlusal height 844, and two of the three center position parameters 816, such as the location along the mesial-distal axis and the location along the root- occlusal height. Other parameters may be modified based on different views. Some or all of the parameters may be modified based on additional imagery from the same or different viewpoints.

The process may be repeated for the same tooth from different perspectives, for each of the selected views. This process can be repeated by analyzing the regions of projection of the dentin template from multiple angles and positions and comparing them across multiple viewpoints, the algorithm can systematically update the parameters for each view and/or internation. This iterative refinement process results in an increasingly accurate 3D representation of the dentin’s geometry at each iteration.

Any of the methods described herein may include detecting internal tissue structures using a trained pattern matching agent. The pattern matching agent may be trained on a dataset of intraoral scan images. In general the trained pattern matching agent may be trained to identify and output a contour of a border between a dentin and enamel, the outer surface of the enamel, or other dental structures, and/or an area of dentin and/or enamel in near infrared images.

The trained pattern matching agent may an artificial intelligence agent, including a machine learning agent. The machine learning agent may be a deep learning agent. In some examples, the trained pattern matching agent may be trained neural network. Any appropriate type of neural network may be used, including generative neural networks. The neural network may be one or more of: perceptron, feed forward neural network, multilayer perceptron, convolutional neural network, radial basis functional neural network, recurrent neural network, long short-term memory (LSTM), sequence to sequence model, modular neural network, etc.

In some examples a trained pattern matching agent may be trained using a training data set of a plurality of intraoral scans that have been reviewed and labeled to indicate enamel and dentin areas, contours, borders, etc. In some embodiments, regions that are not dentin or enamel, or other dental structures may have also been labeled. Each of the plurality of scans may include multiple images (having multiple channels, e.g., near IR, white light, etc.) and all or some of the images may depict overlapping regions.

For example, a trained pattern matching agent may be configured to receive an image, such as a near-infrared image. The trained pattern matching agent may then use the input to identify the edges, contours, areas, etc. corresponding to regions of potential in each of a plurality of images. In some examples, separate, but slightly overlapping images (e.g., taken within a predetermined time and/or having similar camera positions within a proximity threshold) may be used by the trained pattern matching agent.

In some examples the trained pattern matching agent may configured as a competitive neural network. For example, the trained pattern matching agent may be trained on both a positive neural network (indicating the locations where dentin and/or enamel are present) in an image) and a negative neural network, e.g., indicating regions that are not dentin or enamel. This configuration may reduce the number of false positives. In some cases the position of the dentin or enamel region and/or edges may be stored (as well as any confidence score, etc.) and/or applied to a 3D model of the surface of the teeth (e.g., constructed from at least the images being analyzed).

To improve the accuracy of any of the trained networks described herein, the network may be trained by negative labeling. For example, an existing AI network may be trained on second datasets the results given to labelers to mark where the algorithm is wrong (e.g., “negative labeling”); this data may then be feed back into the algorithm training to get an improved network with higher accuracy.

In some examples these apparatuses may use the virtual 3D model as another input. The 3D model may be segmented prior to being used as an input and/or may be used unsegmented. In some example the 2D images provided as input may be segmented. Alternatively segmented images and/or the 3D model of the teeth may be used after the trained pattern matching agent has processed the images.

In general, these methods and apparatuses may be used in real time, e.g., while scanning the teeth or shortly after scanning (e.g., during the same session).

Thus, in general, the trained pattern matching agent may be configured to detect dentin and enamel areas and/or edges from each image and may be configured to indicate one or more geometric properties, such as location, width, 3D geometric model, etc.

### Inputs into the system/method

In any of these methods and apparatuses (e.g., devices and systems, including software, hardware and/or firmware) the input for dentin and enamel detection may be based on an intraoral scan of the patient’s teeth. In particular, the input may be part of an intraoral scanner that includes a near-infrared (near-IR or NIR) scanning as well as visible light (e.g., red, green, blue, or RGB) imaging and/or 3D models of the teeth. In some cases the scanner may also include one or more additional wavelengths, including fluorescent wavelength(s), and/or a viewfinder wavelength. For example, see U.S. 10,507,087, incorporated herein by reference in its entirety. The intraoral scanner may concurrently or sequentially (in an alternating manner) scan one or more images at each of these wavelengths and may build the 3D model. The camera positions may be recorded during each scan. Scans of multiple different wavelengths may be combined or integrated into a single 2D image or a set of 2D channels referred to as a single image. Each image may cover approximately the same region of the oral cavity.

Alternatively or in addition, tooth metadata, such as tooth number, anterior/posterior, crown/original tooth/permanent or deciduous tooth, can be used also as input to the trained pattern matching agent.

In some examples the pattern matching agent may be trained with several images as input. For example, a convolutional neural network (CNN) may be used on each image separately and then the results may be combined by using a fully connected network. The images may be projected to common planes and used as an input to the pattern matching agent. In some examples, a produce volumetric field may be used by using, for example, NERF and the nerf weights may be used as an input to the network.

Any of the methods and apparatuses described herein may use combinations of scan images (e.g., composite images) rather, particularly in instances where the individual images may have relatively small fields of view. Thus, any of the methods and apparatuses described herein may be used with images in which two or more images having adjacent and/or slightly overlapping fields of view may be combined to form a single image. Multiple channels may be used, as described above.

Alternatively or in addition to the use of intraoral scan data (2D images) as described above, in some examples 2D images may be provided by remote scanning using 2D images taken of a patient’s dentition using, e.g., a mobile phone or other imaging device that is not necessarily an intraoral scanner. In some cases, remote scanning (e.g., using a mobile phone) may be used in combination with a 3D model generated by intraoral scanner. In some cases 2D images taken by a mobile phone using an attachment for imaging in both color images and near-infrared (NIR) images may be used. This may allow the patient to self-monitor and take images of these locations. This may further allow tracking by the patient and/or dental professional using the methods and apparatuses described herein. For example, images taken using this technique may be aligned or referenced relative to an existing 3D model.

### Identifying Images Most Likely to Include Dentin and/or Enamel

As mentioned, also described herein are methods and apparatuses (e.g., intraoral scanners) that are configured to identify which images of an intraoral scan, including which near infrared, are most likely to show dentin and/or enamel. These methods and apparatuses may used to rank images of a scan, typically after the scan has been completed, though in some cases the method may be performed in an ongoing manner, as scans are being collected. The ranking indicates the likelihood of an image (e.g., a 2d image from the scan) showing dentin and/or enamel even without having to actually identify dentin and/or enamel in the image(s). The resulting rankings may be used to display images for manual review, e.g., by a user, and/or for automatic detection of dentin and/or enamel, as described above.

Near-infrared images, in particular, may be difficult to interpret given the complexity of the way in which light traverses the interior of the tooth, resulting in different signal behaviors for near infrared images. As a result, dentin and/or enamel may be visible in some images but not readily visible in other images of the same tooth surface. Surprisingly, the ability to visualize dentin and/or enamel in near infrared images may be predicted based on properties of the tooth (tooth surface) and imaging system (e.g., camera and illumination source) and the relationship between the two. The methods and apparatuses described herein may therefore score or rank images from a scan (e.g., an intraoral scan) based on these properties in order to identify a subset of images form the intraoral scan that are most likely to show visible dentin and/or enamel. Thus, some near infrared images are more likely to allow dentin and/or enamel to be seen in the image than others. In one example, the near infrared images from a plurality of near infrared images may be processed as described herein in order to determine a likelihood that a dentin and/or enamel would be visible from that particular near infrared image; this prediction may be based on one or more features describing a relationship between one or more reference points in an internal structure of a tooth in the near infrared image and one or more camera parameters of a camera that is used to take the near infrared image. This may be achieved by identifying, for each image of the plurality of near infrared images, one or more reference points within internal structure of a tooth, and for each one or more reference point, identifying a normal to the reference point relative to the tooth, and a camera angle of the camera taking the near infrared images, and estimating an angle between the normal and the camera angle. In other examples, the accuracy of the prediction may be increased by increasing the parameters or properties used to score the image, beyond just the angle between the normal and the camera angle, as will be described in greater detail herein. In this first example, the property that is used to determine a score of how likely a dentin and/or enamel will be visible in the near infrared image may be based on the camera angle relative to a surface normal of the tooth.

Because there can be hundreds, or even thousands of near-infrared (near infrared) images taken as part of a single scan, it would be extremely helpful to select a subset of images that are most likely to show dentin and/or enamel based on a likelihood score, as described herein. The scored images may be used to determine which images should be used to detect dentin and/or enamel in any of the algorithms described herein, such as in methods 200, 300, 400, and 500, or elsewhere.

Different scoring techniques may be used. For example, a first scoring technique may be based on tooth orientation in relation to the near infrared light source and/or camera position, as mentioned above. Alternatively or additionally, the scoring may be instead based on a trained machine learning agent that uses multiple inputs or properties which may be taken or measured from the image(s). For example, a machine learning agent trained as a classifier may be used by training on properties such as geometrical features of the tooth/tooth internal structures and grey level or other pixel value information from the near infrared images.

In general, there methods and apparatuses may be configured to grade the near infrared images to determine a likelihood that these images will show visible dentin and/or enamel in the near infrared image. In cases where the images having a high likelihood of showing dentin and/or enamel do not include dentin and/or enamel, this may provide a useful negative control. In general, grading the near infrared images with a likelihood for each image that it will show dentin and/or enamel may be more efficient, e.g., more quickly performed, and may be performed as part of the apparatus requiring less time and computational power than analyzing these images to identify actual or potential dentin and/or enamel. Further, even in cases in which dentin and/or enamel may be present, it may be difficult to see the dentin and/or enamel in some images, given the lighting/position of the dentin and/or enamel. Thus, these methods may be performed in combination with any of the dentin and/or enamel detection techniques discussed above, in order to show to the user the best image (e.g., the images most likely to include a visible dentin and/or enamel. In some cases the score/grading mechanism may be used as part of an entirely local process that may be part of the intraoral scanner itself, and may not require a remote processor.

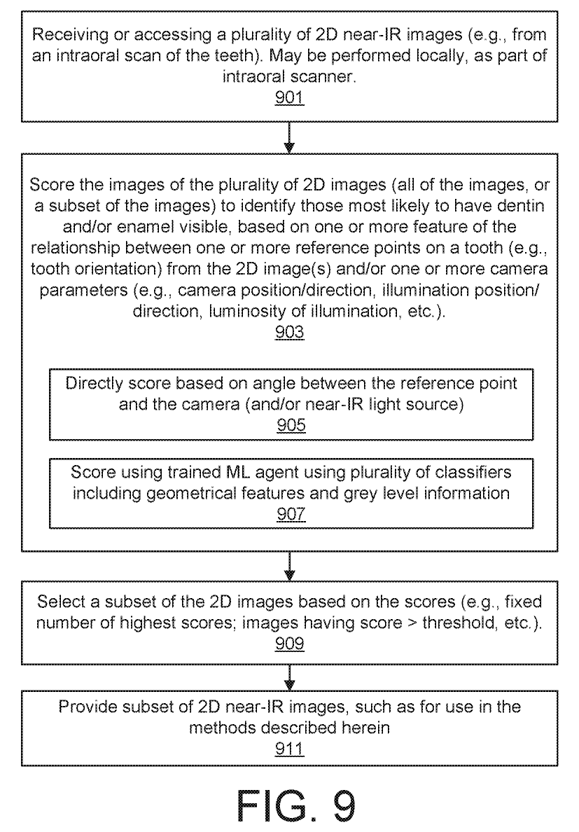

For example, FIG. 9 illustrates a schematic example of a method of identifying near infrared images that are most likely to show a dentin and/or enamel. As shown in FIG. 9, The method may include receiving (and/or accessing) a plurality of 2D near infrared images, e.g., from an intraoral scan of the subject’s dentition at block 901 The processor(s) performing this method may be part of the intraoral scanner (e.g., may be local). As mentioned, because this technique may be performed quickly and requires low computational power, particularly as compared with methods requiring identification of actual dentin and/or enamel, it may be readily performed as part of the intraoral scanner.

The images may then be scored at block 903. All or a subset of the images of the plurality of 2D images may be scored to identify those most likely to have dentin and/or enamel visible. Scoring may be relative to an absolute scale (e.g., regardless of the scores of the other images) or may be relative to the other images from the scan. In some cases the scores may be normalize to the other images. In general, scoring may be based on one or more feature of the relationship between one or more reference points on a tooth (e.g., tooth orientation) from the 2D image(s) and/or one or more camera parameters (e.g., camera position/direction, illumination position/direction, luminosity of illumination, etc.). The images to be scored may be the original images, or they may be modified from the original scan (e.g., filtered, smoothed, interpolated, etc.).

Prior to scoring, the images may be prepared. For example as each image is scored, it may be analyzed, filtered, etc. In some cases the images may be first examined to identify the one or more reference points, e.g., within the tooth structure. For example, one or more reference points may be identified on each tooth, such as a reference point from the distal (e.g., distal interproximal) interproximal region and/or a reference point from the medial (e.g., medial interproximal) region of each tooth. While reference is made to interproximal regions, other regions of the tooth may be used, for example, a location on the tooth surface were the toot surface is perpendicular to the a plane defined by the mesial-distal and root-occlusal axis or defined by the mesial-distal and buccal-lingual axis. The reference points may be identified using the 3D model of the teeth (e.g. digital model, such as a digital surface model of the subject’s dentition). The one or more reference points may be mapped (in some cases from the 3D digital model) to the 2D image(s), such as the near infrared images.

In some cases scoring may be performed by determining, for each of the one or more reference points present in the 2D image, using one particular feature that is well-correlated with the presence and/or absence of dental dentin and/or enamel. For example, scoring may include directly scoring the images based on angle between the reference point and the camera (and/or near infrared light source) at block 905. In this case, the feature is the relationship, e.g., the angle, between a normal on the tooth surface at the reference point, providing tooth orientation, and the camera angle (e.g., a camera parameters that includes the direction of the camera).

Alternatively in some cases multiple different parameters may be used, including some that relate more specifically to the relationship between the tooth (tooth orientation) and the imaging system (e.g., camera, lighting, etc.) or relate to the imaging system (e.g., relationship between the camera and the light source(s)), and/or the tooth. These multiple parameters may be used as classifiers for use with a trained machine learning agent (e.g., algorithm) at block 907. For example a trained ML agent may use a plurality of classifiers including geometrical features and grey level information to determine a score. Any number of different classifiers may be used. As a non-limiting example, the classifiers that may be used may include one or more of: cosine of the angle between camera and normal at reference point, Z absolute value of reference point to camera vector; camera to reference point distance in mm; cosine of the angle between camera direction and reference point; absolute of z value of camera direction vector; cosine of the angle between camera refraction and tooth axis; absolute z value of light source (e.g., LED) direction vector; cosine of the angle between a first light source (e.g., LED1) and normal; cosine of the angle between LED1 direction and reference point; distance between LED1 location and reference point in mm; distance between LED1 location and camera in mm; LED1 luminosity; absolute of z value of reference point to LED1 vector; cosine of the angle between a second light source (e.g., LED2) direction and normal; LED2 luminosity; absolute of z value of reference point to LED2 vector; cosine of the angle between LED2 direction and reference point; distance between LED2 location and reference point in mm; and/or distance between LED2 location and camera in mm.

Once scored, the scores may be adjusted (e.g., normalized) and may be used to filter or otherwise sort the images and may be used to generate one or more sub-sets of images. For example, these methods may select a subset of the 2D images based on the scores. The higher scores may correspond to a greater likelihood of seeing a dental dentin and/or enamel on the image. In general, the higher scoring images may be displayed to the user (e.g., doctor, clinician, dentist, orthodontist, etc.). The higher scoring images may be selected for display and/or saving (e.g., as a subset of the total images in the scan) 909. In some cases those images having a score that is greater than a minimum threshold may be displayed. If scores as between 0 and 1.0 or any other range, the threshold may be adjusted so that only the highest x percent (e.g., 90%, 95%, 96%, 97%, 98%, 99%, 99.1%, etc.) are shown or selected for display. In some case the threshold may be set and/or adjusted by the user (e.g., in a user display/interface, the user may select, or adjust, this threshold up/down). In some cases the method or apparatus may simply display the top y number of most likely e.g., a fixed number of highest scores; images having score > threshold, etc.). Alternatively or additionally, the higher-scoring images may be displayed at block 911. Thus, in any of these examples a separate selection step, 909, is not required, but instead the scored images may be directly displayed, e.g., in a user interface.

The scores may be stored (e.g., in a data field, as metadata, etc.) and/or an index of the scores including the corresponding images or reference to uniquely identify the images, may be stored locally and securely by or within the intraoral scanner and/or a remote server. As mentioned here, any appropriate user interface and display may be used, including showing the high-scoring images individually or as a group or groups. The user may switch between different images, manually or automatically. In some cases, the user may select region or teeth (e.g., on a 3D model/display, including a 3D surface model) and be shown a 2D image having a high score that corresponds to the selected tooth or teeth (ore region). The 2D image may include markings indicating the presence of a high-scoring 2D image that is or can be, displayed. In some cases, images that do not have a sufficiently high score (e.g., below a threshold) may not be shown, and/or may be “discarded” from the user interface. Alternatively, in some cases the user may select to specifically be shown some or all of the lower-scoring images.

As mentioned, in some cases the method or apparatus may use the angle between a representative point and the camera and/or light source as a parameter, either on its own or as a parameter input into a trained machine learning agent. For example, the method or apparatus may be configured to compute a score that is based on the angle between a normal to the surface of the tooth at the selected representative tooth and the primary transmission angle of a light source emitting the light used to capture the image (e.g., one or more LED that is used for transmitting the near infrared light to the teeth). In some cases multiple light sources may be used. The location of the light from the light source and the position of light in relation to the possible dentin and/or enamel may impact the visibility of the dentin and/or enamel. Thus the score may be implemented based on an angle that is formed between the normal and the source of light from the LED; for example, the smaller the angle, the higher the score. In cases where very close or near images are scored similarly high, near-duplicate or very similar and/or overlapping images may be removed or marked as duplicate (and may not need to be displayed). In some cases only on high-scoring image (e.g., highest scoring image) may be selected and/or displayed.

As mentioned, any of these methods and apparatuses may include determining a representative or reference point, also referred to as an anchor point, that may be used as a basis of calculation. Since the location of an actual dentin and/or enamel is not known, these reference points may be broadly selected as areas where the dentin and/or enamel may be, for example, within the internal structure of a tooth. This reference point may be used to determine a normal (surface normal) from the tooth.

Embodiments as disclosed and/or described herein can be implemented in the form of control logic using computer software in a modular or integrated manner. Based on the disclosure and teachings provided herein, a person of ordinary skill in the art will know and appreciate other ways and/or methods to implement the present invention using hardware and a combination of hardware and software.

The disclosure includes the following clauses and embodiments:

Clause 1. A method for use in dental treatment planning, the method comprising: receiving or collecting a plurality of two-dimensional (2D) near infrared intraoral scanner images of a patient’s tooth and three-dimensional (3D) surface data of the patient’s tooth; identifying, for each image of the plurality of 2D intraoral scanner images, a border between a dentin and an enamel in the images using a detection algorithm that uses the plurality of 2D near infrared images; projecting the border between the dentin and the enamel identified in each image onto a three-dimensional (3D) model of the patient’s tooth, wherein the 3D model of the patient’s tooth is generated from 3D surface data of the patient’s tooth; detect an outer edge of the tooth relative to the border between a dentin and an enamel based on a location of the outer edge of the tooth of the 3D model in the projection; determining an enamel width for the patient’s tooth based on the projected border; and outputting an indicator of enamel width.

Clause 2. The method of clause 1, wherein the detection algorithm is an edge detection algorithm.

Clause 3. The method of clause 1, wherein the detection algorithm is a trained pattern matching agent that is trained to use the plurality of 2D near infrared image.

Clause 4. The method of clause 1, wherein outputting comprises outputting a 3D model of the patient’s teeth including the enamel width projected on the 3D model.

Clause 5. The method of clause 1, further comprising segmenting the 3D model of the patient’s teeth before projecting the border between the dentin and the enamel on the 3D model.

Clause 6. The method of clause 1, wherein receiving or collecting the plurality of 2D intraoral scanner images of the patient’s teeth comprises receiving the plurality of 2D intraoral scanner images from an intraoral scanner data set.

Clause 7. The method of clause 1, further comprising: receiving an indication of one or more views for detecting enamel width.

Clause 8. The method of clause 7, further comprising: determining which of the plurality of 2D images corresponds to the one or more views.

Clause 9. The method of clause 8, wherein identifying, for each image of the plurality of 2D intraoral scanner images, a border between a dentin and an enamel in the images using a detection algorithm that uses the plurality of 2D near infrared images, includes identifying for each of the plurality of 2D images that corresponds to the one or more views, a border between a dentin and an enamel in the images using a detection algorithm that uses the plurality of 2D near infrared images.

Clause 10. The method of clause 9, wherein projecting the border between the dentin and the enamel identified in each image onto a three-dimensional (3D) model of the patient’s tooth, includes projecting the border between the dentin and the enamel identified in each image that corresponds to the one or more views onto a three-dimensional (3D) model of the patient’s tooth.

Clause 11. The method of clause 10, wherein determining an enamel width for the patient’s tooth based on the projected border includes determining the enamel width based on the projected borders between the dentin and the enamel for one or more views.

Clause 12. A method for use in dental treatment planning, the method comprising: receiving or collecting a plurality of two-dimensional (2D) near infrared intraoral scanner images of a patient’s tooth and three-dimensional (3D) surface data of the patient’s tooth; identifying, for each image of the plurality of 2D intraoral scanner images, a border between a dentin and an enamel in the images using a detection algorithm that uses the plurality of 2D near infrared images; projecting the border between the dentin and the enamel identified in each image onto a three-dimensional (3D) model of the patient’s tooth, wherein the 3D model of the patient’s tooth is generated from 3D surface data of the patient’s tooth; correcting the location of the border between the enamel and the dentin by tracing rays between a modeled camera and the 3D model of the tooth; generating a 3D model of the dentin based on the projection; and outputting the 3D model of the dentin.

Clause 13. The method of clause 12, wherein the detection algorithm is an edge detection algorithm.

Clause 14. The method of clause 12, wherein the detection algorithm is a trained pattern matching agent that is trained to use the plurality of 2D near infrared image.

Clause 15. The method of clause 12, wherein outputting comprises outputting a 3D model of the patient’s teeth including the 3D model of the dentin projected on the 3D model of the patient’s tooth.

Clause 16. The method of clause 12, further comprising segmenting the 3D model of the patient’s teeth before projecting the border between the dentin and the enamel on the 3D model.

Clause 17. The method of clause 12, wherein receiving or collecting the plurality of 2D intraoral scanner images of the patient’s teeth comprises receiving the plurality of 2D intraoral scanner images from an intraoral scanner data set.

Clause 18. The method of clause 12, further comprising: receiving an indication of a plurality of views for detecting dentin geometry.

Clause 19. The method of clause 18, further comprising: determining which of the plurality of 2D images corresponds to the one or more views.

Clause 20. The method of clause 19, wherein identifying, for each image of the plurality of 2D intraoral scanner images, a border between a dentin and an enamel in the images using a detection algorithm that uses the plurality of 2D near infrared images, includes identifying for each of the plurality of 2D images that corresponds to the one or more views, a border between a dentin and an enamel in the images using a detection algorithm that uses the plurality of 2D near infrared images.

Clause 21. The method of clause 20, wherein projecting the border between the dentin and the enamel identified in each image onto a three-dimensional (3D) model of the patient’s tooth, includes projecting the border between the dentin and the enamel identified in each image that corresponds to the one or more views onto a three-dimensional (3D) model of the patient’s tooth.

Clause 22. The method of clause 21, wherein determining a dentin geometry for the patient’s tooth based on the projected border includes determining the dentin geometry based on the projected borders between the dentin and the enamel for plurality of views.

Clause 23. The method of clause 22, wherein the plurality of views are orthogonal to each other.

Clause 24. A method for use in dental treatment planning, the method comprising: receiving or collecting a plurality of two-dimensional (2D) near infrared intraoral scanner images of a patient’s tooth and three-dimensional (3D) surface data of the patient’s tooth; receiving a parameterized dentin template; identifying, for each image of the plurality of 2D intraoral scanner images, a border between a dentin and an enamel in the images using a detection algorithm that uses the plurality of 2D near infrared images; projecting the border between the dentin and the enamel identified in each image onto a three-dimensional (3D) model of the patient’s tooth, wherein the 3D model of the patient’s tooth is generated from 3D surface data of the patient’s tooth; iteratively projecting the dentin template on the 3D model for each of the 2D images until the projected template aligns with the border between the dentin and the enamel identified in each image; generating a 3D model of the dentin based on the projection; and outputting the 3D model of the dentin.

Clause 25. The method of clause 24, wherein the detection algorithm is an edge detection algorithm.

Clause 26. The method of clause 24, wherein the detection algorithm is a trained pattern matching agent that is trained to use the plurality of 2D near infrared image.

Clause 27. The method of clause 24, wherein the template is a generic dentin template.

Clause 28. The method of clause 24, wherein the template is the 3D model of the patient’s tooth.

Clause 29. The method of clause 24, wherein generating a 3D model of the dentin based on the projection includes modifying the parameters of the dentin template.

Clause 30. The method of clause 24, wherein outputting comprises outputting a 3D model of the patient’s teeth including the 3D model of the dentin projected on the 3D model of the patient’s tooth.

Clause 31. The method of clause 24, further comprising segmenting the 3D model of the patient’s teeth before projecting the border between the dentin and the enamel on the 3D model.

Clause 32. The method of clause 24, wherein receiving or collecting the plurality of 2D intraoral scanner images of the patient’s teeth comprises receiving the plurality of 2D intraoral scanner images from an intraoral scanner data set.

Clause 33. The method of clause 24, further comprising: receiving an indication of a plurality of views for detecting dentin geometry.

Clause 34. The method of clause 24, further comprising: determining which of the plurality of 2D images corresponds to the one or more views.

Clause 35. The method of clause 34, wherein identifying, for each image of the plurality of 2D intraoral scanner images, a border between a dentin and an enamel in the images using a detection algorithm that uses the plurality of 2D near infrared images, includes identifying for each of the plurality of 2D images that corresponds to the one or more views, a border between a dentin and an enamel in the images using a detection algorithm that uses the plurality of 2D near infrared images.

Clause 36. The method of clause 35, wherein projecting the border between the dentin and the enamel identified in each image onto a three-dimensional (3D) model of the patient’s tooth, includes projecting the border between the dentin and the enamel identified in each image that corresponds to the one or more views onto a three-dimensional (3D) model of the patient’s tooth.

Clause 37. The method of clause 36, wherein determining a dentin geometry for the patient’s tooth based on the projected border includes determining the dentin geometry based on the projected borders between the dentin and the enamel for plurality of views.

Clause 38. The method of clause 37, wherein the plurality of views include a occlusal view and a buccal or lingual view.

Clause 39. The method of clause 24, further comprising: correcting the location of the border between the enamel and the dentin by tracing rays between a modeled camera and the 3D model of the tooth.

Clause 40. A system for use in dental treatment planning, the system comprising: one or more processors; and memory comprising instructions that when executed by the one or more processors causes the system to carry out the method of any one of clause 1 – 39.The software components, processes, or functions disclosed and/or described in this application may be implemented as software code to be executed by a processor using a suitable computer language such as Python, Java, JavaScript, C, C++, or Perl using conventional or object-oriented techniques. The software code may be stored as a series of instructions or commands in (or on) a non-transitory computer-readable medium, such as a random-access memory (RAM), a read only memory (ROM), a magnetic medium such as a hard-drive, or an optical medium such as a CD-ROM. In this context, a non-transitory computer-readable medium is a medium suitable for the storage of data or an instruction set aside from a transitory waveform. Such computer readable medium may reside on or within a single computational apparatus and may be present on or within different computational apparatuses within a system or network.

According to one example implementation, the term processing element or processor, as used herein, may be a central processing unit (CPU), or conceptualized as a CPU (such as a virtual machine). In this example implementation, the CPU or a device in which the CPU is incorporated may be coupled, connected, and/or in communication with one or more peripheral devices, such as a display. In another example implementation, the processing element or processor may be incorporated into a mobile computing device, such as a smartphone or tablet computer.

The non-transitory computer-readable storage medium referred to herein may include a number of physical drive units, such as a redundant array of independent disks (RAID), a flash memory, a USB flash drive, an external hard disk drive, thumb drive, pen drive, key drive, a High-Density Digital Versatile Disc (HD-DV D) optical disc drive, an internal hard disk drive, a Blu-Ray optical disc drive, or a Holographic Digital Data Storage (HDDS) optical disc drive, synchronous dynamic random access memory (SDRAM), or similar devices or forms of memories based on similar technologies. Such computer-readable storage media allow the processing element or processor to access computer-executable process steps and application programs, stored on removable and non-removable memory media, to off-load data from a device or to upload data to a device. As mentioned, with regards to the embodiments disclosed and/or described herein, a non-transitory computer-readable medium may include a structure, technology, or method apart from a transitory waveform or similar medium.

Example embodiments of the disclosure are described herein with reference to block diagrams of systems, and/or flowcharts or flow diagrams of functions, operations, processes, or methods. One or more blocks of the block diagrams, or one or more stages or steps of the flowcharts or flow diagrams, and combinations of blocks in the block diagrams and combinations of stages or steps of the flowcharts or flow diagrams may be implemented by computer-executable program instructions. In some embodiments, one or more of the blocks, or stages or steps may not necessarily need to be performed in the order presented or may not necessarily need to be performed at all.

The computer-executable program instructions may be loaded onto a general-purpose computer, a special purpose computer, a processor, or other programmable data processing apparatus to produce a specific example of a machine. The instructions that are executed by the computer, processor, or other programmable data processing apparatus create means for implementing one or more of the functions, operations, processes, or methods disclosed and/or described herein. The computer program instructions may be stored in (or on) a computer-readable memory that may direct a computer or other programmable data processing apparatus to function in a specific manner, such that the instructions stored in (or on) the computer-readable memory produce an article of manufacture including instruction means that when executed implement one or more of the functions, operations, processes, or methods disclosed and/or described herein.

While embodiments of the disclosure have been described in connection with what is presently considered to be the most practical approach and technology, the embodiments are not limited to the disclosed implementations. Instead, the disclosed implementations are intended to include and cover modifications and equivalent arrangements included within the scope of the appended claims. Although specific terms are employed herein, they are used in a generic and descriptive sense only and not for purposes of limitation.

This written description uses examples to describe one or more embodiments of the disclosure, and to enable a person skilled in the art to practice the disclosed approach and technology, including making and using devices or systems and performing the associated methods. The patentable scope of the disclosure is defined by the claims, and may include other examples that occur to those skilled in the art. Such other examples are intended to be within the scope of the claims if they have structural and/or functional elements that do not differ from the literal language of the claims, or if they include structural and/or functional elements with insubstantial differences from the literal language of the claims.

All references, including publications, patent applications, and patents, cited herein are hereby incorporated by reference to the same extent as if each reference was individually and specifically indicated to be incorporated by reference and/or was set forth in its entirety herein.

The use of the terms “a” and “an” and “the” and similar references in the specification and in the claims are to be construed to cover both the singular and the plural, unless otherwise indicated herein or clearly contradicted by context. The terms “having,” “including,” “containing” and similar references in the specification and in the claims are to be construed as open-ended terms (e.g., meaning “including, but not limited to,”) unless otherwise noted.

Recitation of ranges of values herein are intended to serve as a shorthand method of referring individually to each separate value inclusively falling within the range, unless otherwise indicated herein, and each separate value is incorporated into the specification as if it were individually recited herein. Method steps or stages disclosed and/or described herein may be performed in any suitable order unless otherwise indicated herein or clearly contradicted by context.

The use of examples or exemplary language (e.g., “such as”) herein, is intended to illustrate embodiments of the disclosure and does not pose a limitation to the scope of the claims unless otherwise indicated. No language in the specification should be construed as indicating any non-claimed element as essential to each embodiment of the disclosure.

As used herein (i.e., the claims, figures, and specification), the term “or” is used inclusively to refer items in the alternative and in combination.

Different arrangements of the elements, structures, components, or steps illustrated in the figures or described herein, as well as components and steps not shown or described are possible. Similarly, some features and sub-combinations are useful and may be employed without reference to other features and sub-combinations. Embodiments have been described for illustrative and not for restrictive purposes, and alternative embodiments may become apparent to readers of the specification. Accordingly, the disclosure is not limited to the embodiments described in the specification or depicted in the figures, and modifications may be made without departing from the scope of the appended claims.

One or more embodiments of the disclosed subject matter are described herein with specificity to meet statutory requirements, but this description does not limit the scope of the claims. The claimed subject matter may be embodied in other ways, may include different elements or steps, and may be used in conjunction with other existing or later developed technologies. This description should not be interpreted as implying any required order or arrangement among or between various steps or elements except when the order of individual steps or arrangement of elements is explicitly noted as being required.

Embodiments of the disclosure will be described more fully herein with reference to the accompanying drawings, which form a part hereof, and which show, by way of illustration, exemplary embodiments by which the disclosure may be practiced. The disclosure may, however, be embodied in different forms and should not be construed as limited to the embodiments set forth herein; rather, these embodiments are provided so that this disclosure will satisfy the statutory requirements and convey the scope of the disclosure to those skilled in the art.

Among others, the subject matter of the disclosure may be embodied in whole or in part as a system, as one or more methods, or as one or more devices. Embodiments may take the form of a hardware implemented embodiment, a software implemented embodiment, or an embodiment combining software and hardware aspects. For example, in some embodiments, one or more of the operations, functions, processes, or methods described herein may be implemented by one or more suitable processing elements (such as a processor, microprocessor, co-processor, CPU, GPU, TPU, QPU, or controller, as non-limiting examples) that is part of a client device, server, network element, remote platform (such as a SaaS platform), an “in the cloud” service, or other form of computing or data processing system, device, or platform.

The processing element or elements may be programmed with a set of executable instructions (e.g., software instructions), where the instructions may be stored in (or on) one or more suitable non-transitory data storage elements. In some embodiments, the set of instructions may be conveyed to a user through a transfer of instructions or an application that executes a set of instructions (such as over a network, e.g., the Internet). In some embodiments, a set of instructions or an application may be utilized by an end-user through access to a SaaS platform or a service provided through such a platform.

In some embodiments, the systems and methods disclosed herein may provide services through a SaaS or multi-tenant platform. The platform provides access to multiple entities, each with a separate account and associated data storage. Each account may correspond to a dentist or orthodontist, a patient, an entity, a set or category of entities, a set or category of patients, an insurance company, or an organization, for example. Each account may access one or more services, a set of which are instantiated in their account, and which implement one or more of the methods or functions disclosed and/or described herein.

In some embodiments, one or more of the operations, functions, processes, or methods described herein may be implemented by a specialized form of hardware, such as a programmable gate array, application specific integrated circuit (ASIC), or the like. Note that an embodiment of the inventive methods may be implemented in the form of an application, a sub-routine that is part of a larger application, a “plug-in”, an extension to the functionality of a data processing system or platform, or other suitable form. The following detailed description is, therefore, not to be taken in a limiting sense.

1. A method for use in dental treatment planning, the method comprising:
collecting, with an intraoral scanner, a plurality of two-dimensional (2D) near infrared intraoral scanner images of a patient’s tooth and three-dimensional (3D) surface data of the patient’s tooth; and
using a model of a dentin of a tooth of a patient to administer dental treatment, wherein the dental treatment includes providing a physical dental prosthetic, wherein the physical dental prosthetic is based on a 3D model of a dentin of the tooth of the patient,
wherein the 3D model of the dentin is based on a parameterized dentin template,
wherein, for each image of the plurality of 2D near infrared images, a border was generated between a dentin and an enamel in the images using a detection algorithm that uses the plurality of 2D near infrared images,
wherein the border was projected between the dentin and the enamel identified in each image onto a three-dimensional (3D) model of the patient’s tooth, wherein the 3D model of the patient’s tooth was generated from 3D surface data of the patient’s tooth,
wherein the dentin template was iteratively projected on the 3D model for each of the 2D near infrared images until the projection aligns with the border between the dentin and the enamel identified in each image, and
wherein the 3D model of the dentin was generated based on the projection.

2. The method of claim , wherein the detection algorithm is an edge detection algorithm.

3. The method of claim , wherein the detection algorithm is a trained pattern matching agent that is trained to use the plurality of 2D near infrared image.

4. The method of claim , wherein the template is a generic dentin template.

5. The method of claim , wherein the template is the 3D model of the patient’s tooth.

6. The method of claim , wherein the parameters of the dentin template are modified to generate the 3D model of the dentin.

7. The method of claim , wherein using a model of a dentin comprises using a 3D model of the tooth of a patient including the 3D model of the dentin projected on the 3D model of the patient’s tooth.

8. The method of claim , wherein a location of the border between the enamel and the dentin was corrected by tracing rays between a modeled camera of the intraoral scanner and the 3D model of the tooth.

9. A system for use in dental treatment planning, the system comprising:
one or more processors; and
memory comprising instructions that when executed by the one or more processors causes the system to carry out a method comprising:
receiving or collecting a plurality of two-dimensional (2D) near infrared intraoral scanner images of a patient’s tooth and three-dimensional (3D) surface data of the patient’s tooth;
identifying, for each image of the plurality of 2D intraoral scanner images, a border between a dentin and an enamel in the images using a detection algorithm that uses the plurality of 2D near infrared images;
projecting the border between the dentin and the enamel identified in each image onto a three-dimensional (3D) model of the patient’s tooth, wherein the 3D model of the patient’s tooth is generated from 3D surface data of the patient’s tooth;
correcting a location of the border between the enamel and the dentin by tracing rays between a modeled camera and the 3D model of the tooth;
generating a 3D model of the dentin based on the projection; and
outputting the 3D model of the dentin.

10. The system of claim , wherein the detection algorithm is an edge detection algorithm.

11. The system of claim , wherein the detection algorithm is a trained pattern matching agent that is trained to use the plurality of 2D near infrared image.

12. The system of claim , wherein outputting comprises outputting a 3D model of the tooth of a patient including the 3D model of the dentin projected on the 3D model of the patient’s tooth.

13. The system of claim , wherein the method further comprises:
segmenting the 3D model of the tooth of a patient before projecting the border between the dentin and the enamel on the 3D model.

14. The system of claim , wherein receiving or collecting the plurality of 2D intraoral scanner images of the tooth of a patient comprises receiving the plurality of 2D intraoral scanner images from an intraoral scanner data set.

15. The system of claim , wherein the method further comprises:
receiving an indication of a plurality of views for detecting dentin geometry.

16. The system of claim , wherein the method further comprises:
determining which of the plurality of 2D intraoral scanner images corresponds to the one or more views.

17. The system of claim , wherein identifying, for each image of the plurality of 2D intraoral scanner images, a border between a dentin and an enamel in the images using a detection algorithm that uses the plurality of 2D near infrared images, includes identifying for each of the plurality of 2D intraoral scanner images that corresponds to the one or more views, a border between a dentin and an enamel in the images using a detection algorithm that uses the plurality of 2D near infrared images.

18. The system of claim , wherein projecting the border between the dentin and the enamel identified in each image onto a three-dimensional (3D) model of the patient’s tooth, includes projecting the border between the dentin and the enamel identified in each image that corresponds to the one or more views onto a three-dimensional (3D) model of the patient’s tooth.

19. The system of claim , wherein determining a dentin geometry for the patient’s tooth based on the projected border includes determining the dentin geometry based on the projected borders between the dentin and the enamel for plurality of views.

20. A system comprising:
a non-transitory computer medium storing computer-program instructions for executing steps of a method of orthodontic treatment of a patient, the method comprising the method of claim 1.
